(19) United States
(12) Patent Application Publication
(10) Pub. No.: US 2016/0128638 A1
(43) Pub. Date: May 12, 2016

(54) SYSTEM AND METHOD FOR DETECTING AND QUANTIFYING DEVIATIONS FROM PHYSIOLOGICAL SIGNALS NORMALITY

(71) Applicant: Bloom Technologies NV, Diepenbeek (BE)

(72) Inventors: Marco Altini, Eindhoven (NL); Julien Penders, Liege (BE); Eric Dy, San Francisco, CA (US); Torfinn Berset, Veldhoven (NL)

(21) Appl. No.: 14/537,458

(22) Filed: Nov. 10, 2014

(51) Int. Cl. A61B 5/00; A61B 5/08; A61B 5/053; A61B 5/0205

(52) U.S. Cl. CPC A61B 5/7275; A61B 5/0205; A61B 5/08; A61B 5/0533

(57) ABSTRACT

A system for detecting and quantifying deviations from physiological signals normality and methods for making and using same. Each subject physiology follows unique patterns. The physiological signals can be affected by one or more factors such as circadian rhythm, disease and/or external stressors. Deviations of physiological signals from the normality of a subject can be indicative of external events that might require proper lifestyle management or just in time interventions, such as being exposed to high stress or the progress/onset of specific disease conditions. The disclosed system advantageously can quantify such deviations.

### FIELD

The present disclosure is related to the field of mobile health and personalized data analytics, and, more specifically, to a method and system for detecting and quantifying deviations from physiological signals normality.

### BACKGROUND

Conventional health-related applications for lifestyle or disease progression monitoring rely on measuring physiological signals of a person to determine whether such physiological signals are within known norms for a specific population. One example of a health-related application can measure physiological signals, such as heart rate, blood pressure, and respiration, to assist a person with maintaining a healthy lifestyle. Monitoring the physiological signals relative to known norms typically is used to prevent upcoming health-related issues, exposure to above-normal stressors requiring proper management or even just in time intervention.

These applications can span from physical to mental health. An example of an application concerning physical health can be cardiorespiratory fitness or energy expenditure estimation. Several applications in both lifestyle management and self-monitoring or quantified self like applications as well as traditional medical applications require remote physiological data monitoring for disease management. Thus a wide spectrum of applications can be covered.

The problem of understanding when physiological signals are outside of the normality of a person or heading in such direction is complex due to multiple factors.

First, an issue arising from the use of physiological data is the context-dependency of such signals. The natural change over periods of twenty-four hours due to the circadian rhythm as well as the influence of factors such as eating, physical activity, the environment etc. make interpretation of physiological data measurements very complex in free living settings. Traditionally, the medical community has been discarding values outside very controlled conditions in order to guarantee the availability of a known context. For example taking measurements always at the same time of the day, typically after waking up in order to limit the influence of other factors. Alternatively, taking a measurement in controlled environment at the hospital or the general practitioner's office. However it is clear from recent studies that continuous physiological data can provide more insights on the health status of a person. Thus, motivating the need for approaches able to aid the interpretation of continuous physiological signals.

Secondly, the problem of determining normal values for different physiological signals in many populations, which can be important in understanding when physiological signals are outside of such norms, might require acquiring data from a high number of persons of different characteristics. Traditionally, such applications making use of physiological signals relied on norms collected in epidemiological studies involving many participants. For example tens or hundreds of thousands. However, even for physiological signals with well-known prognostic values and a long history of use in medical applications, such as blood pressure, these norms are often too broad and not stratified enough, providing limited value. For example while it is well known that blood pressure is lower in women than in men, guidelines do not differ per gender, often causing underestimation of increasing blood pressure in women, since values are still within what are considered norms for the whole population, clearly requiring more resolution in stratifications. For rare conditions the population approach might not even be possible since there might be only a very limited number of people with similar conditions.

Finally, even when specific contexts and norm values are present understanding to what extent the physiological signals of the person diverge from what is considered to be normality, is a further issue. Often deviations are quantified only as differences from known norms, without capturing the degree to which they affect the individual in particular. Being able to quantify context-specific changes in physiological data for a person, as opposed to the population, can provide more precise health assessments.

Recently, machine learning approaches have been used to detect deviations from a living being physiological signals normality for a multitude of applications. However, the current approaches rely on supervised methods which require a significant amount of data from similar subjects to be present, in order to determine norms from which deviations can be measured. This approach suffers from limitations previously mentioned, since physiological data is very personal and supervised population-based model are not accurate enough.

In view of the foregoing, a need exists for an improved system and method for detecting and quantifying deviations from the physiological signals normality of a subject.

### BRIEF DESCRIPTION OF THE DRAWINGS

FIG. 1A is an exemplary top-level flow chart illustrating an embodiment of a method for detecting deviations from physiological signals normality, wherein the method comprises data acquisition, context recognition and physiological signals modeling.

FIG. 1B is an exemplary flow chart illustrating an alternative embodiment of the method of FIG. 1A, wherein the method further includes abnormality detection and quantification.

FIG. 2A is an exemplary flow chart illustrating a preferred embodiment for acquiring data of FIG. 1A or 1B wherein acquiring data includes receiving data from smartphone sensors.

FIG. 2B is an exemplary flow chart illustrating a preferred embodiment for acquiring data of FIG. 1A or 1B wherein acquiring data includes receiving data from smartphone sensors and preprocessing data.

FIG. 2C is an exemplary flow chart illustrating a preferred embodiment for acquiring data of FIG. 1A or 1B wherein acquiring data includes receiving data from wearable sensors.

FIG. 2D is an exemplary flow chart illustrating a preferred embodiment for acquiring data of FIG. 1A or 1B wherein acquiring data includes receiving data from wearable sensors and preprocessing data.

FIG. 2E is an exemplary flow chart illustrating a preferred embodiment for acquiring data of FIG. 1A or 1B wherein acquiring data includes receiving data from smartphone sensors and wearable sensors.

FIG. 2F is an exemplary flow chart illustrating a preferred embodiment for acquiring data of FIG. 1A or 1B wherein acquiring data includes receiving data from smartphone sensors and wearable sensors and preprocessing data.

FIG. 2G is an exemplary flow chart illustrating a preferred embodiment for acquiring data of FIG. 1A or 1B wherein acquiring data includes receiving data from smartphone sensors, wearable sensors and manual input.

FIG. 2H is an exemplary flow chart illustrating a preferred embodiment for acquiring data of FIG. 1A or 1B wherein acquiring data includes receiving data from smartphone sensors, wearable sensors and manual input and preprocessing data.

FIG. 21 is an exemplary flow chart illustrating a preferred embodiment for acquiring data of FIG. 1A or 1B wherein acquiring data includes receiving data from smartphone sensors, wearable sensors, ambient sensors, APIs and manual input.

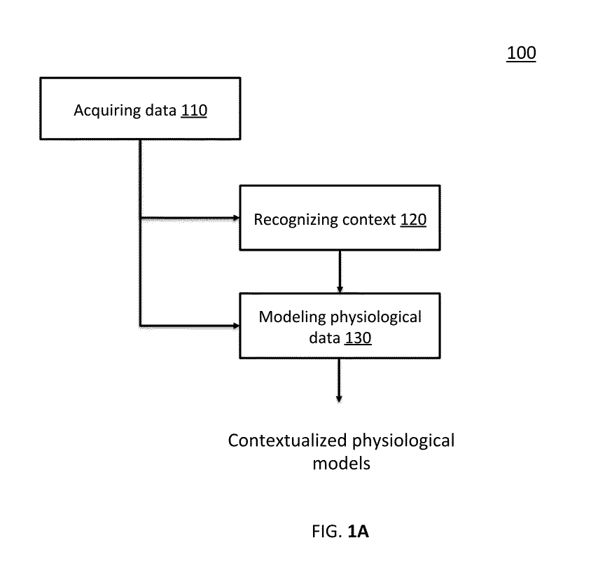

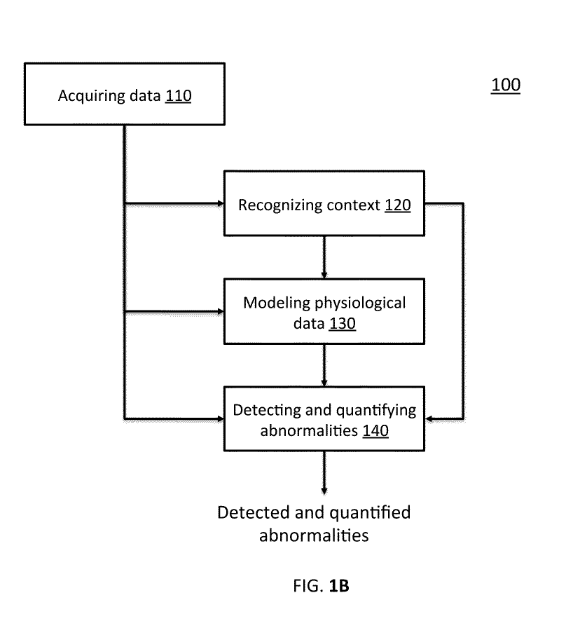

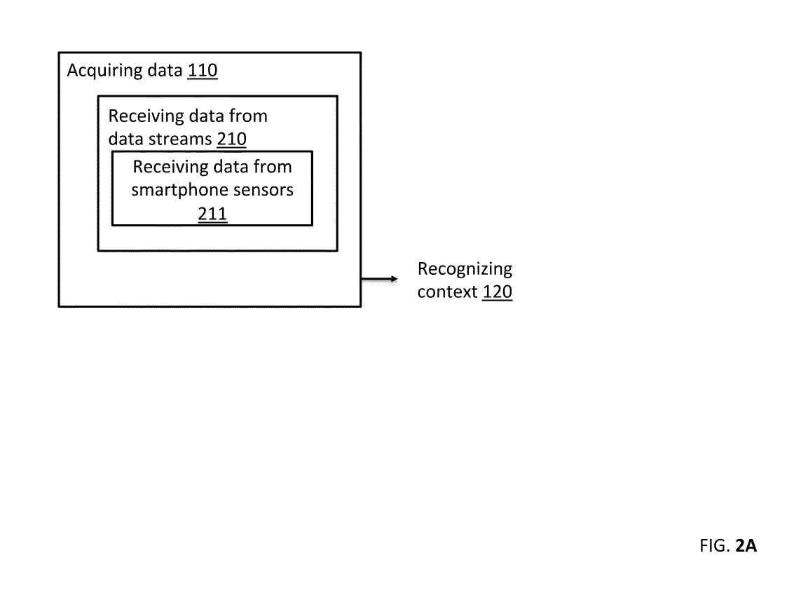

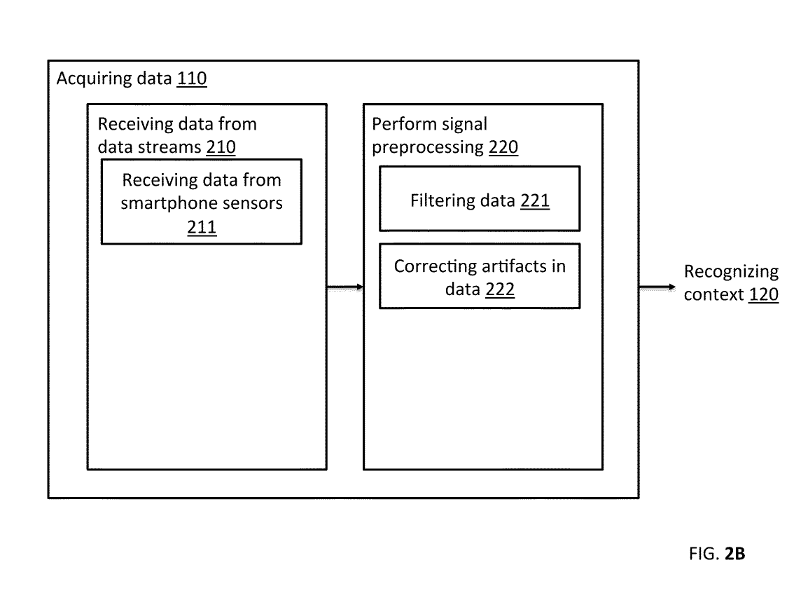

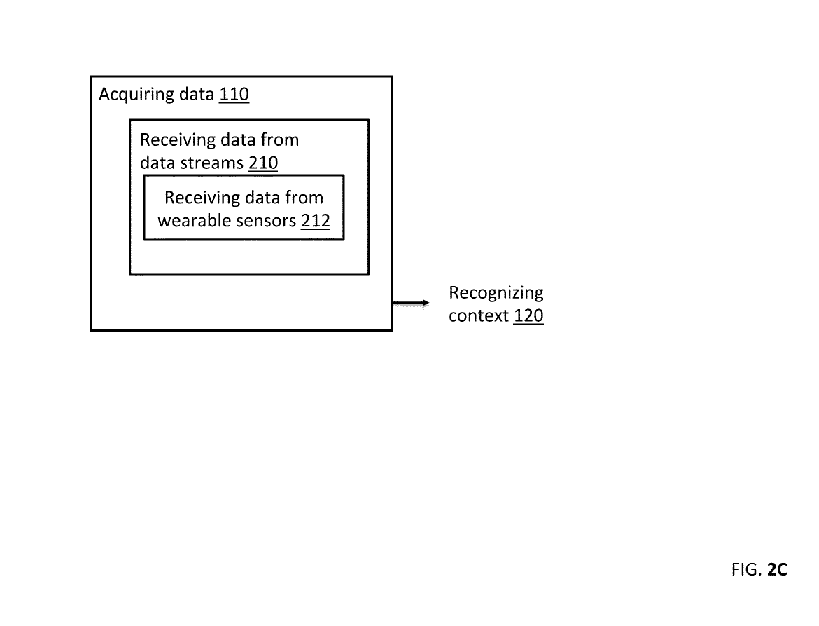

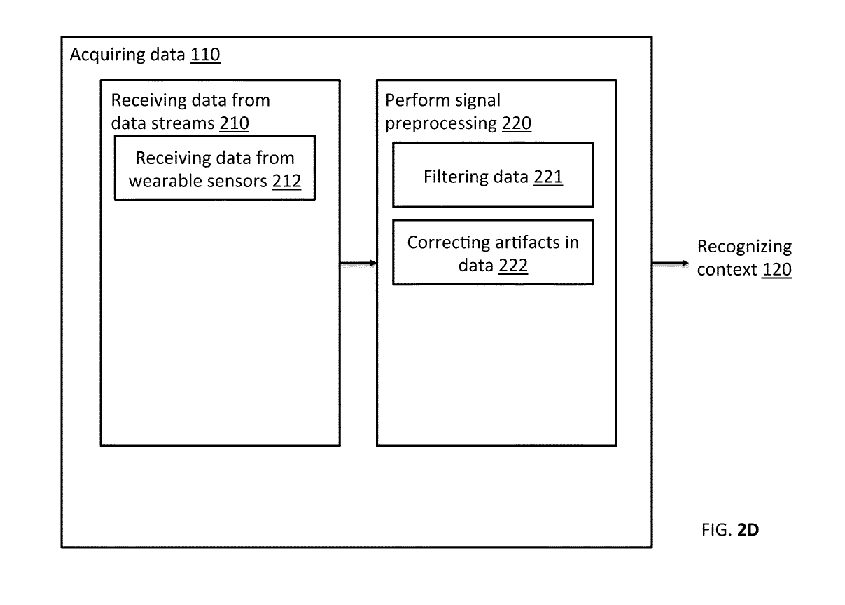

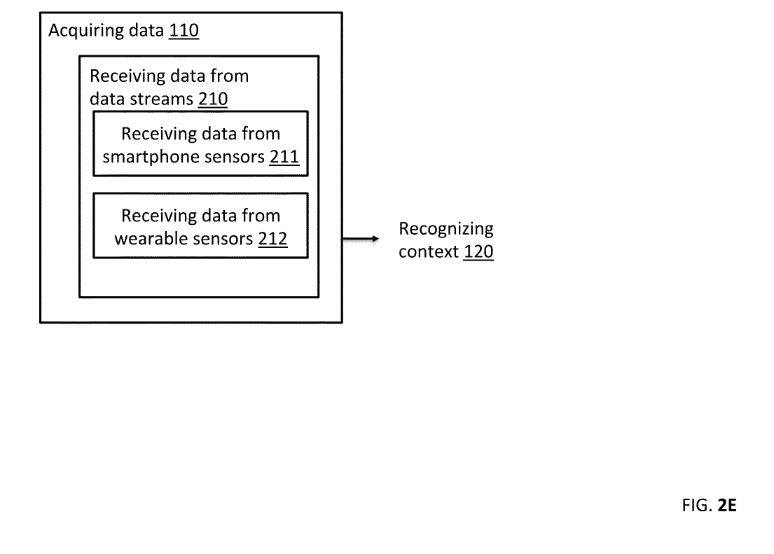

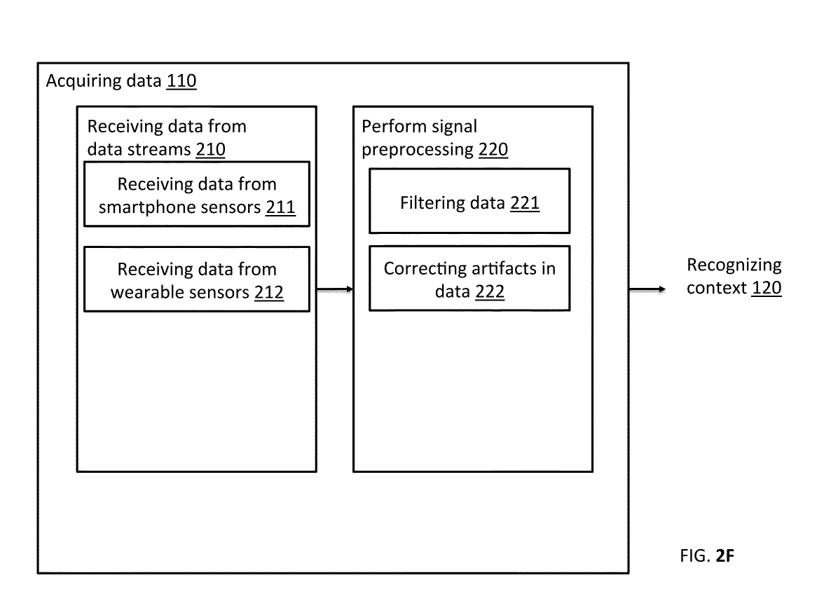

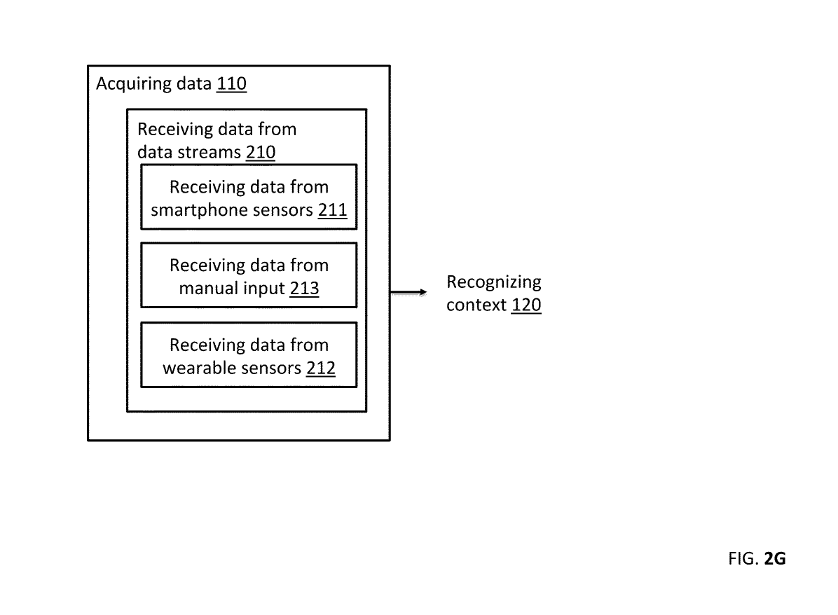

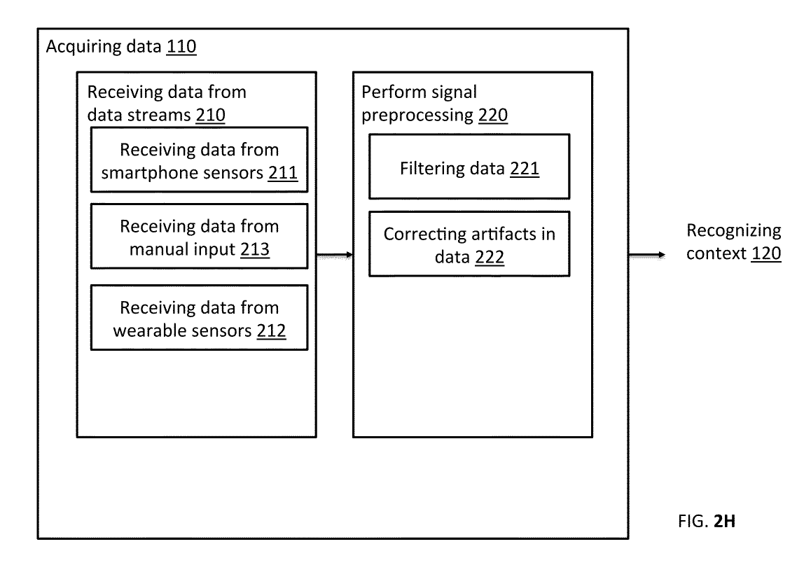

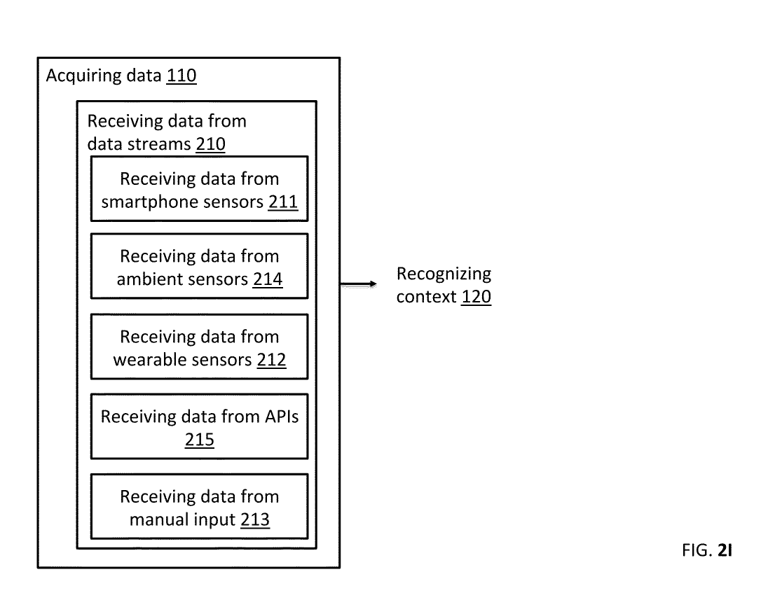

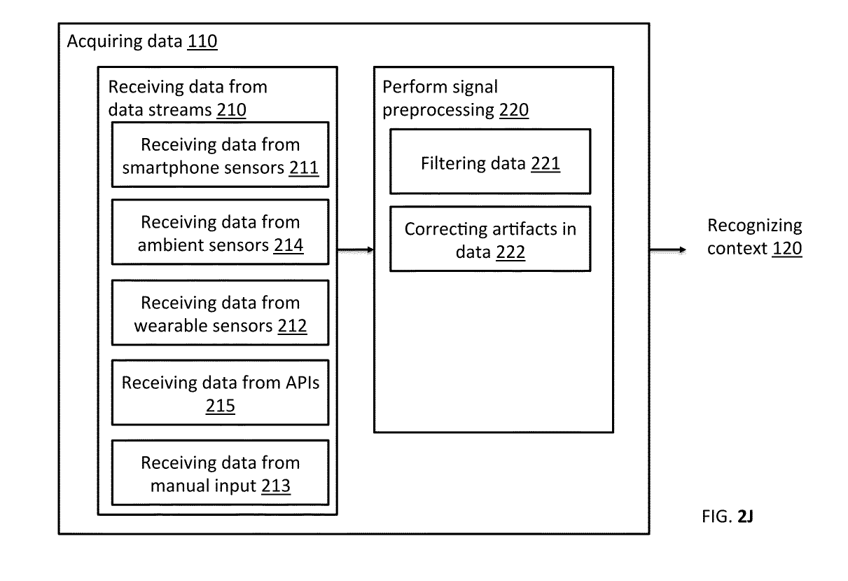

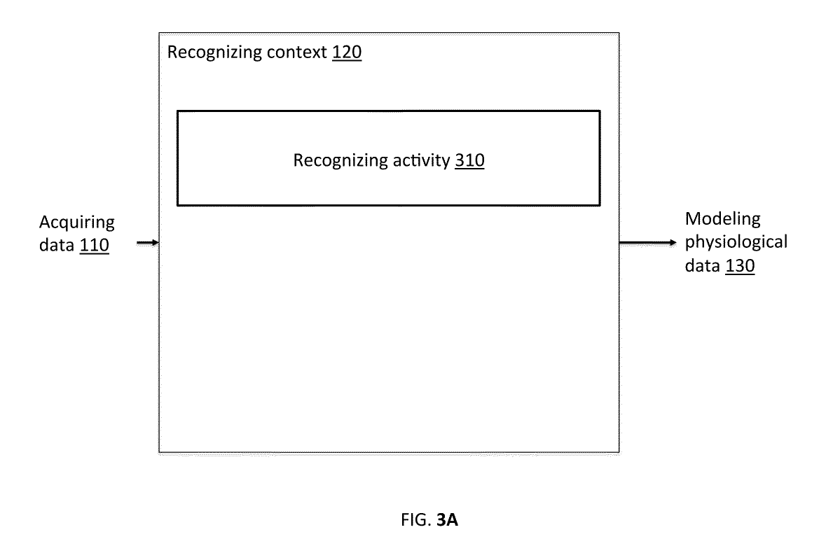

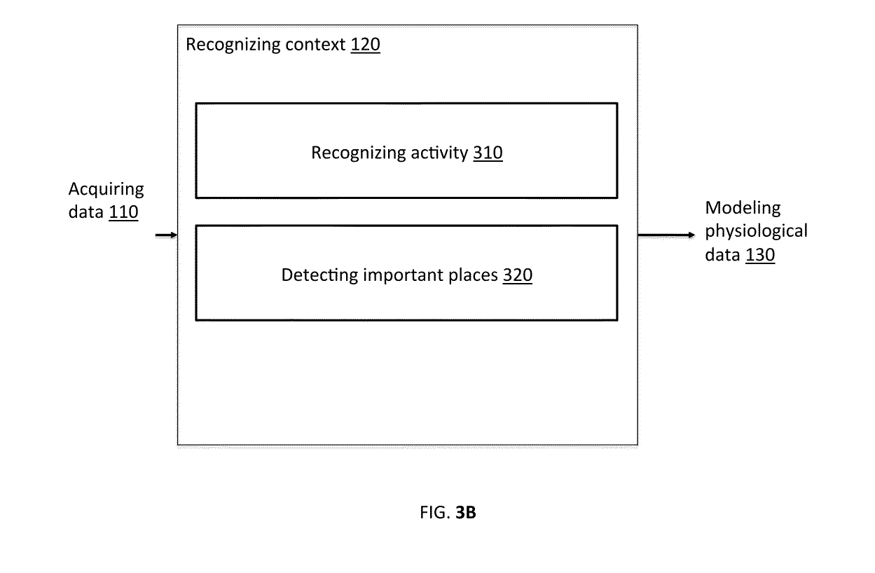

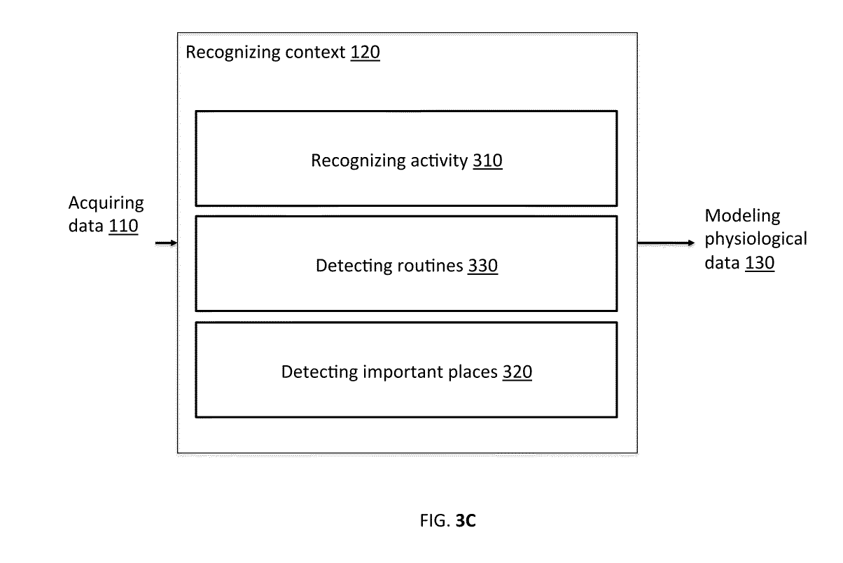

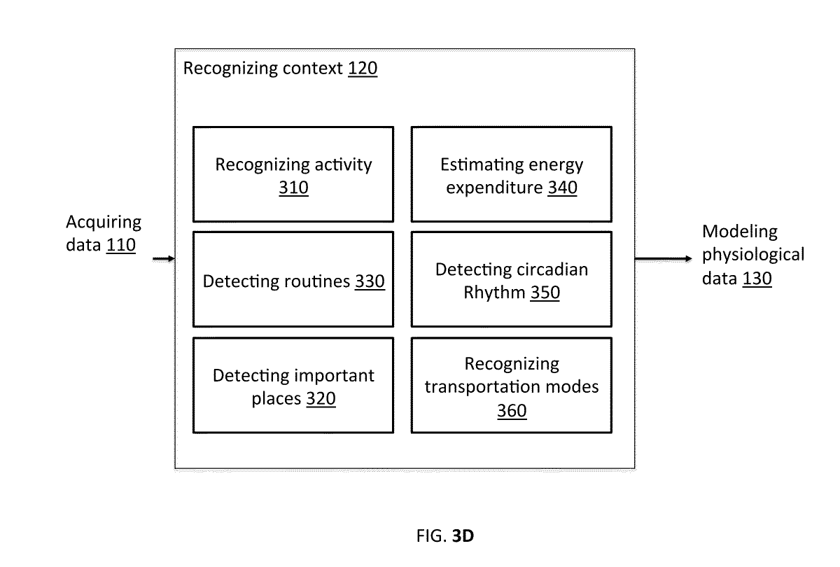

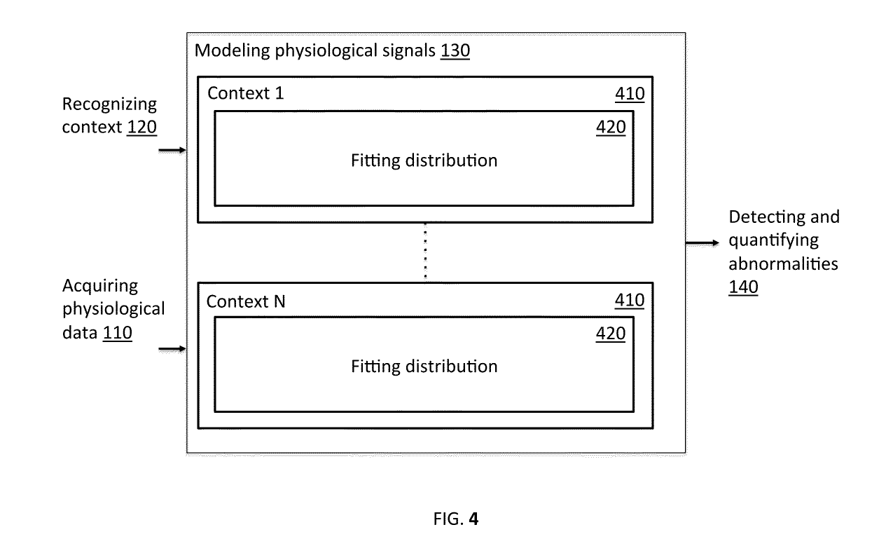

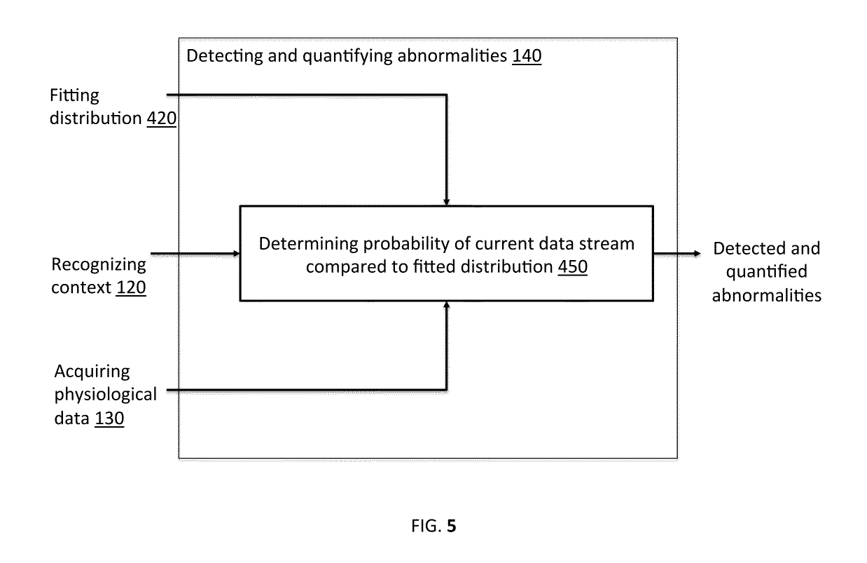

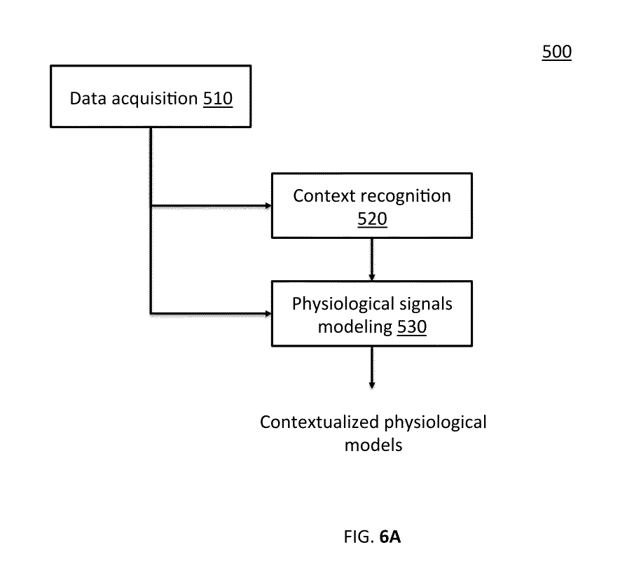

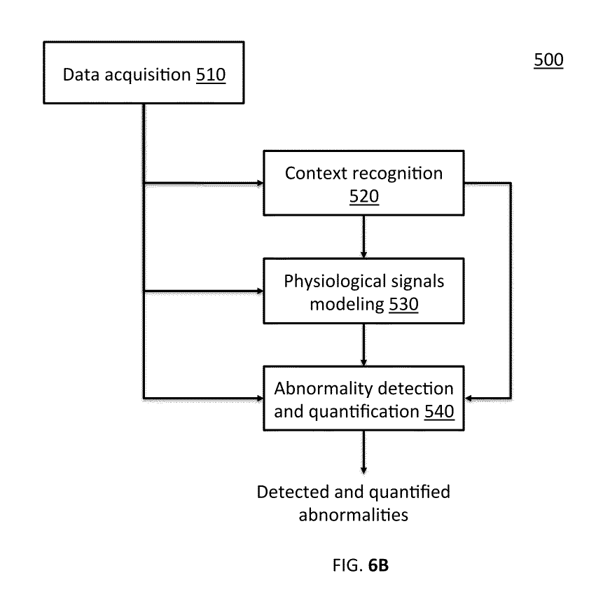

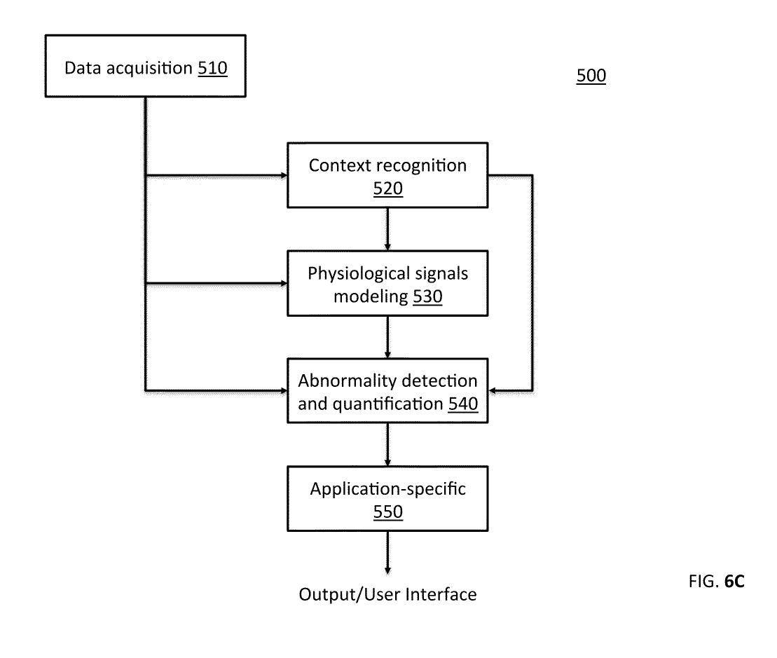

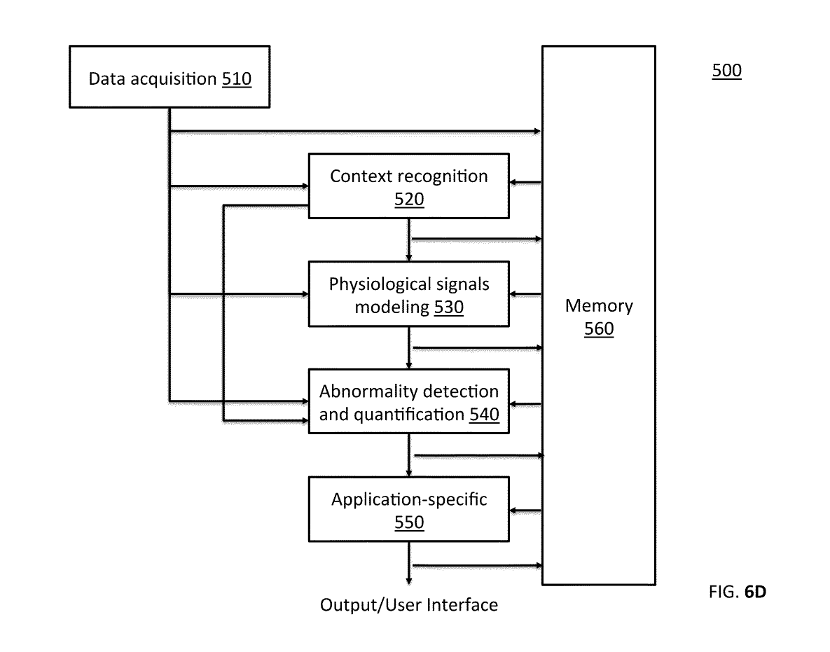

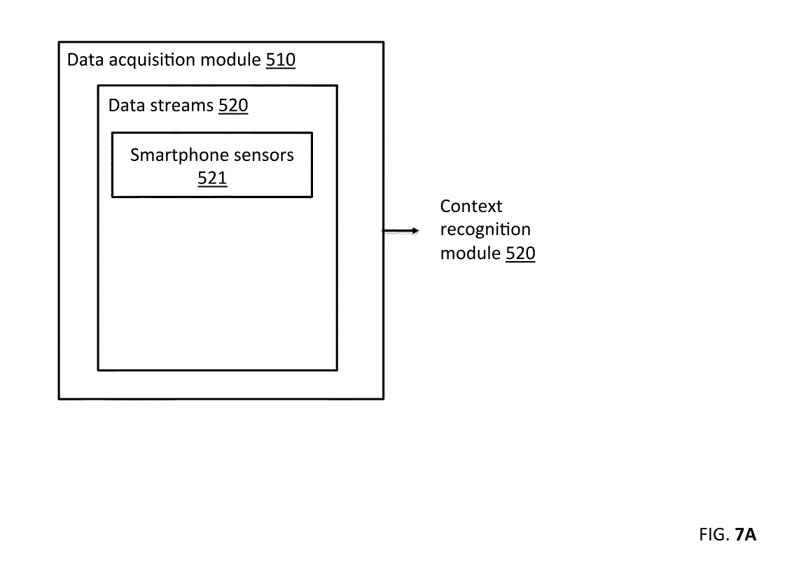

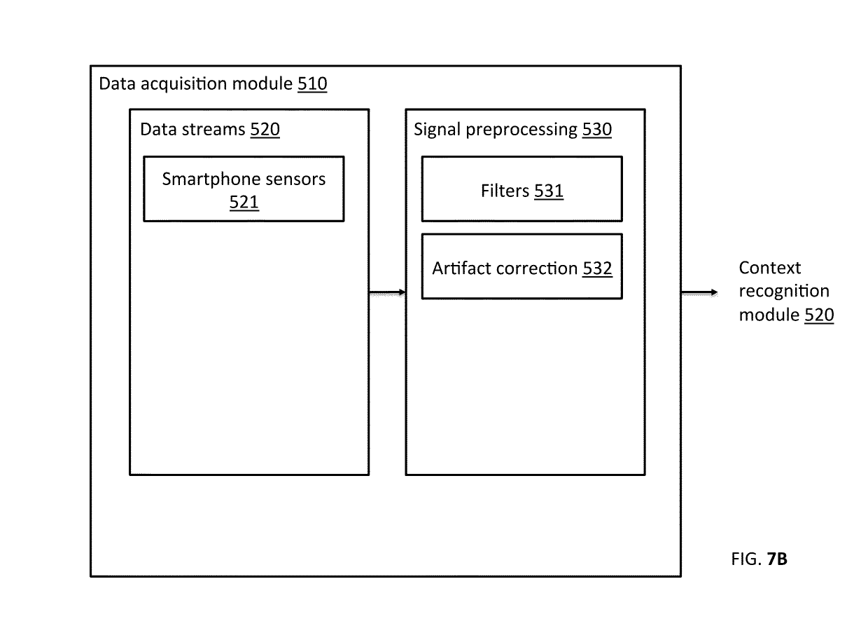

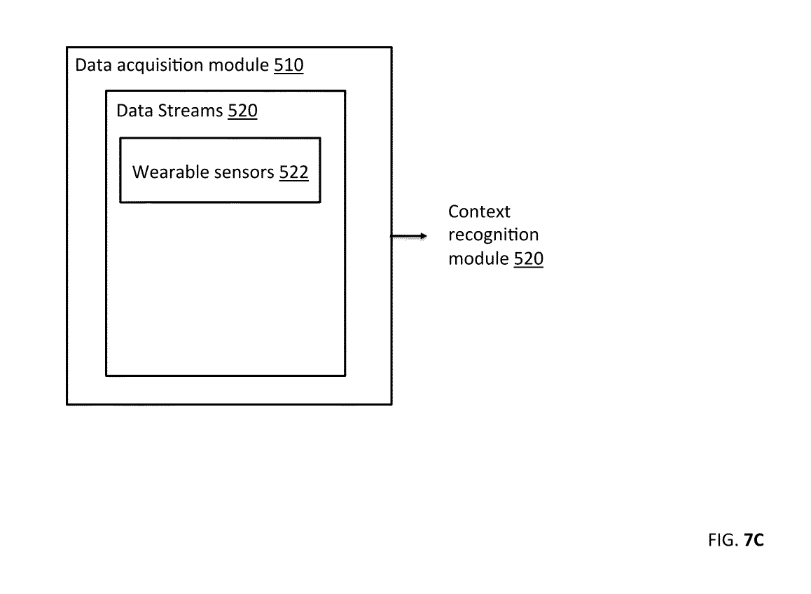

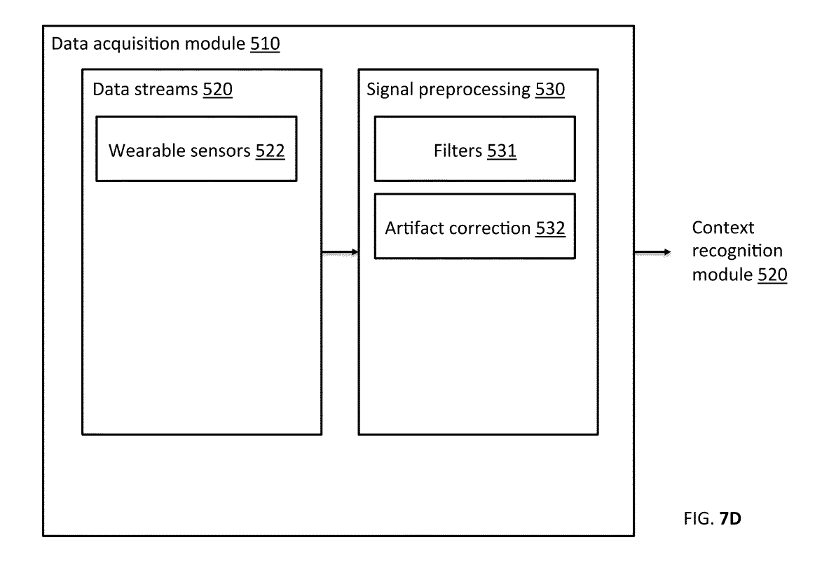

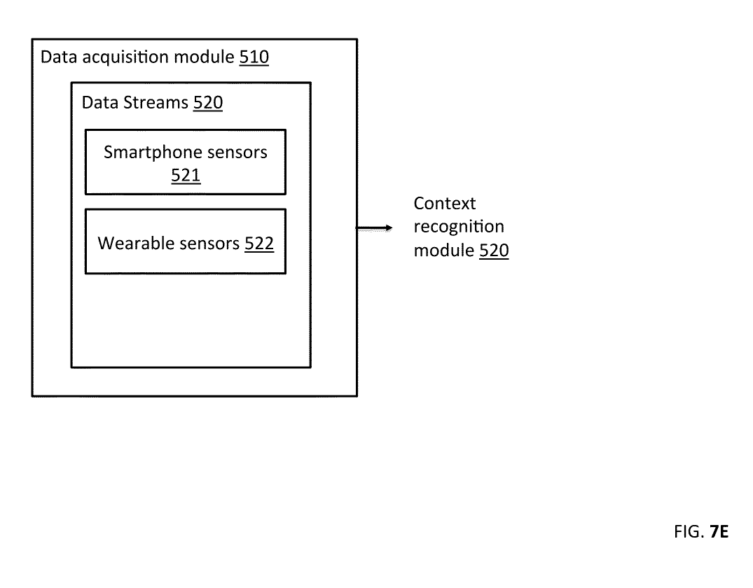

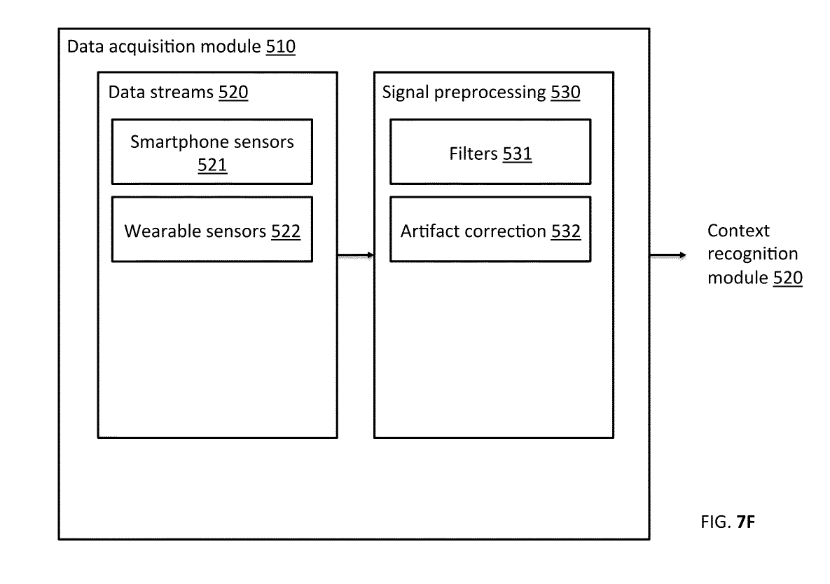

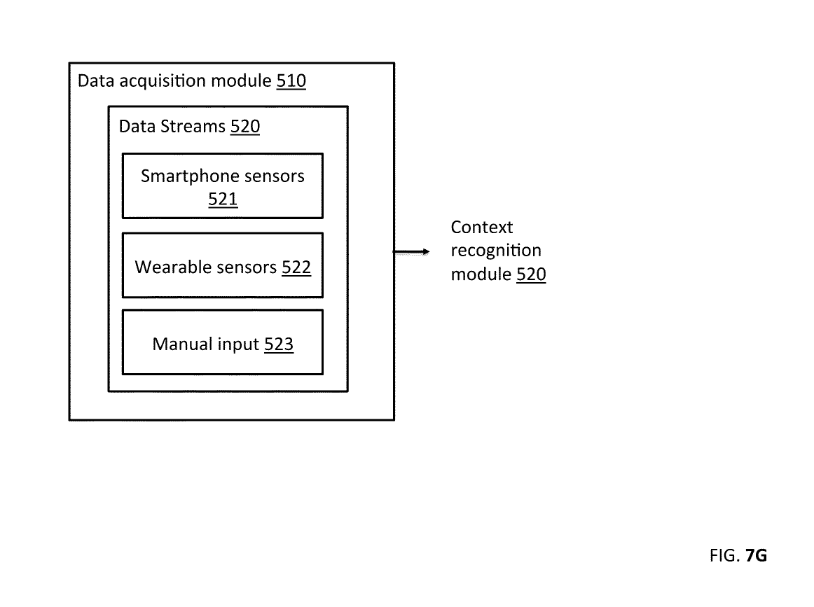

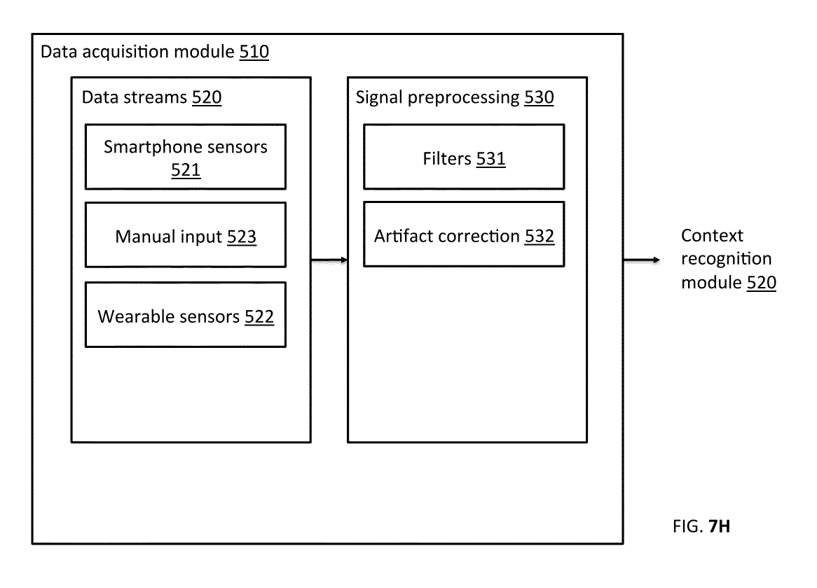

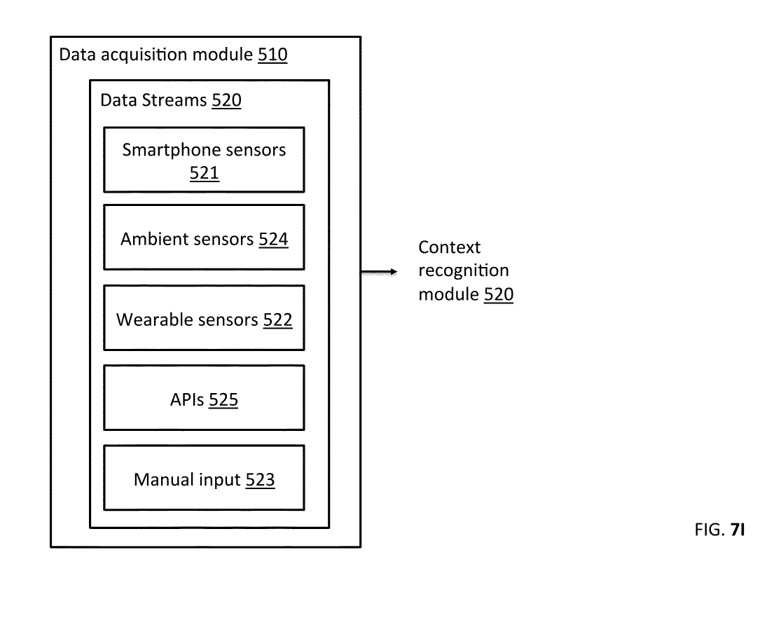

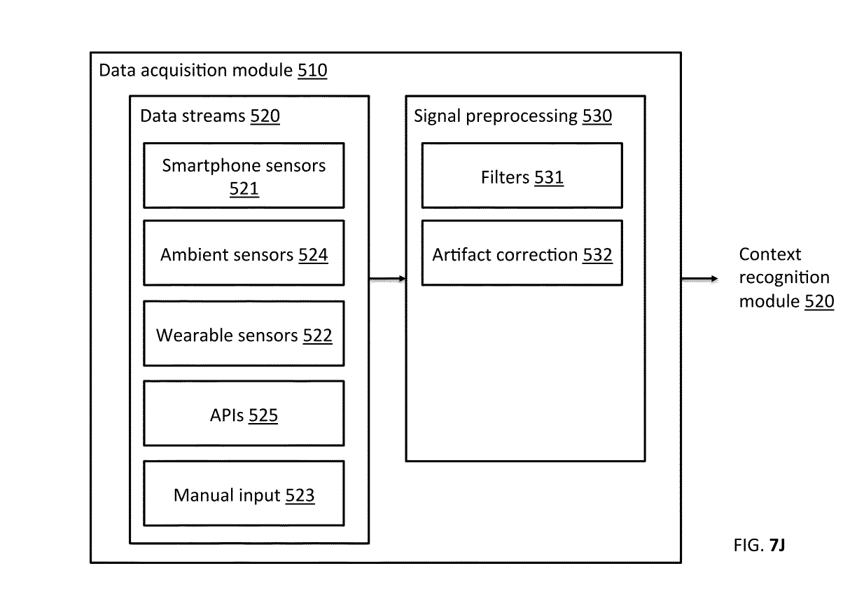

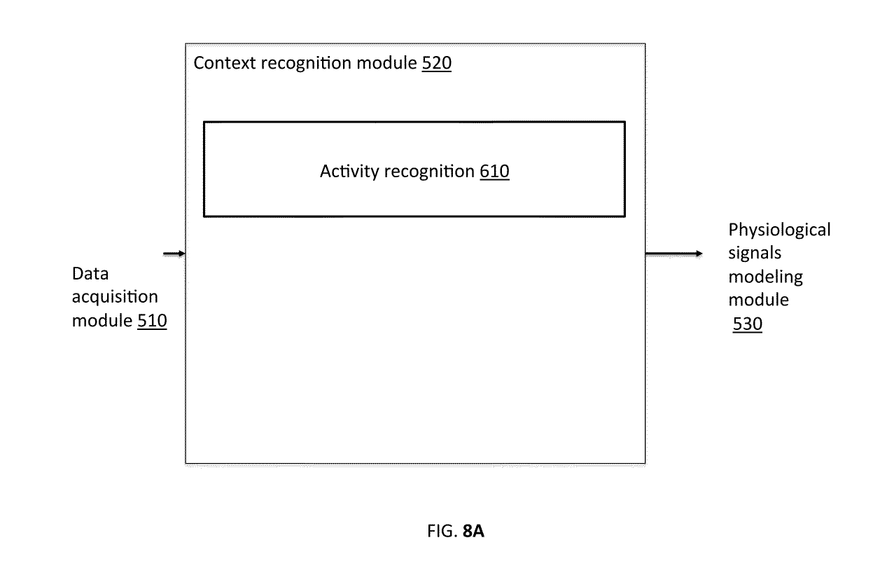

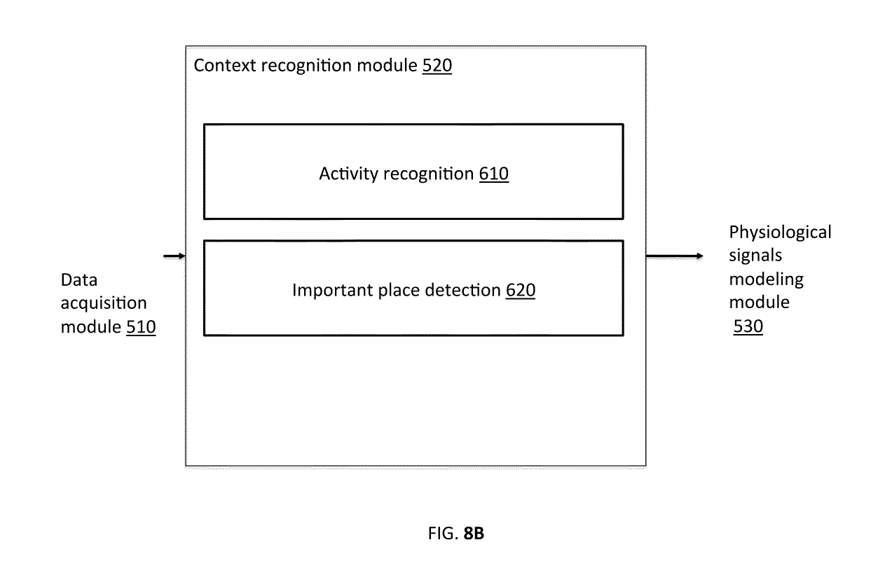

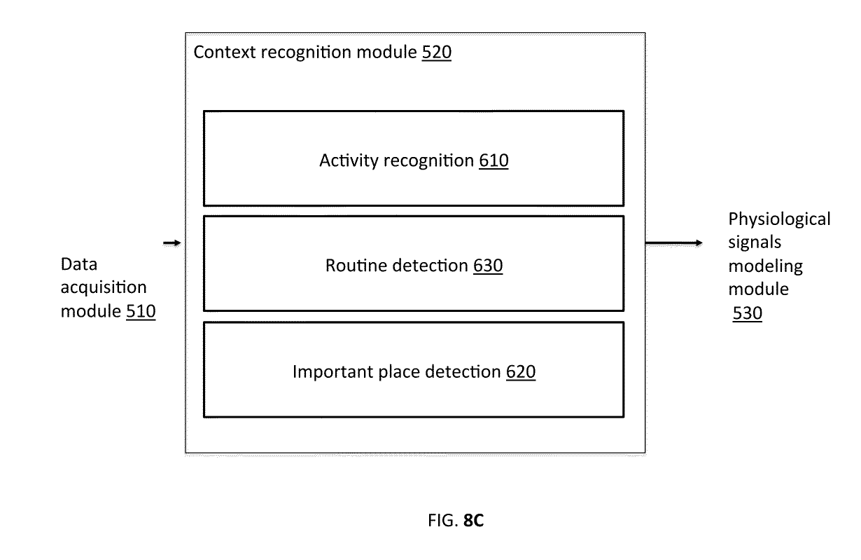

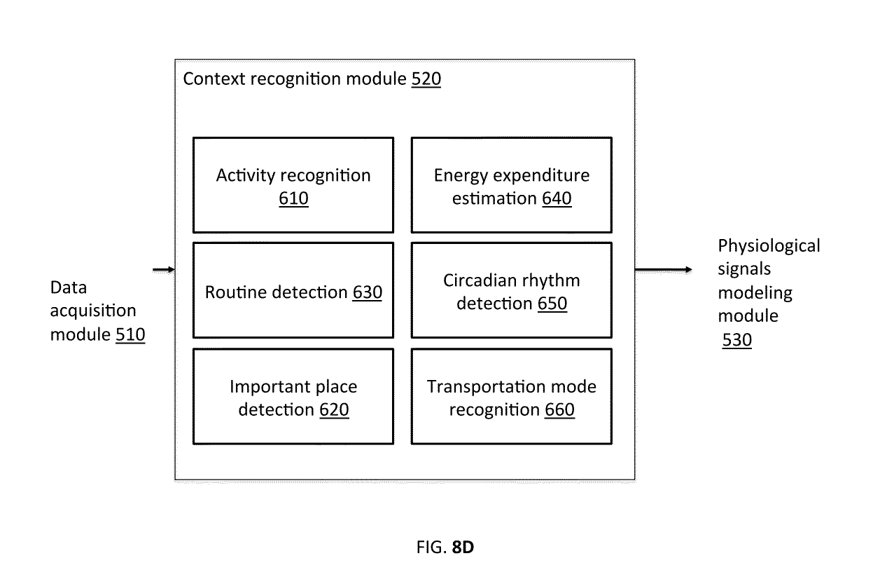

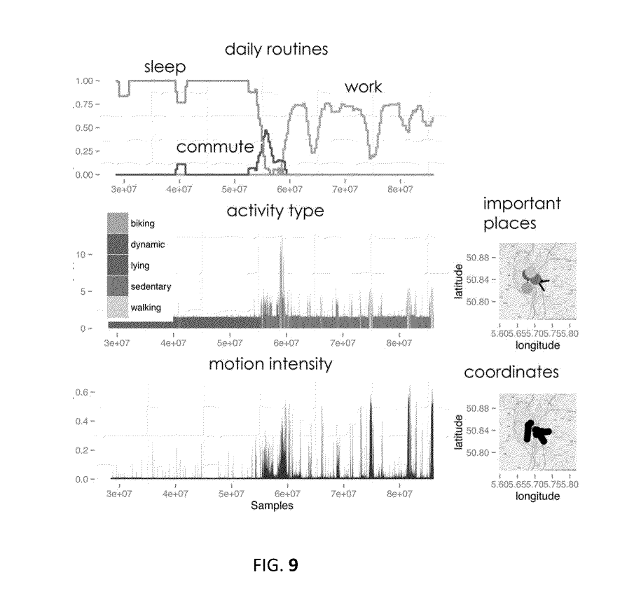

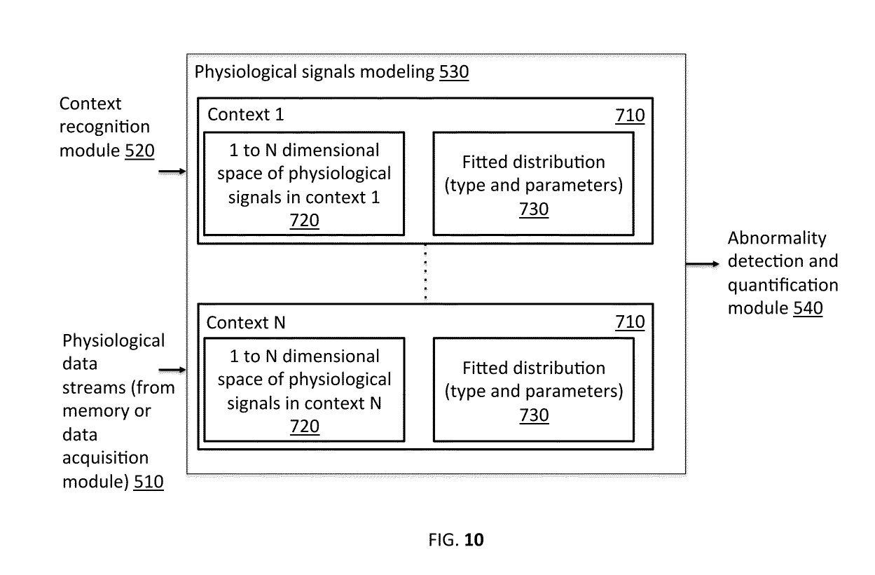

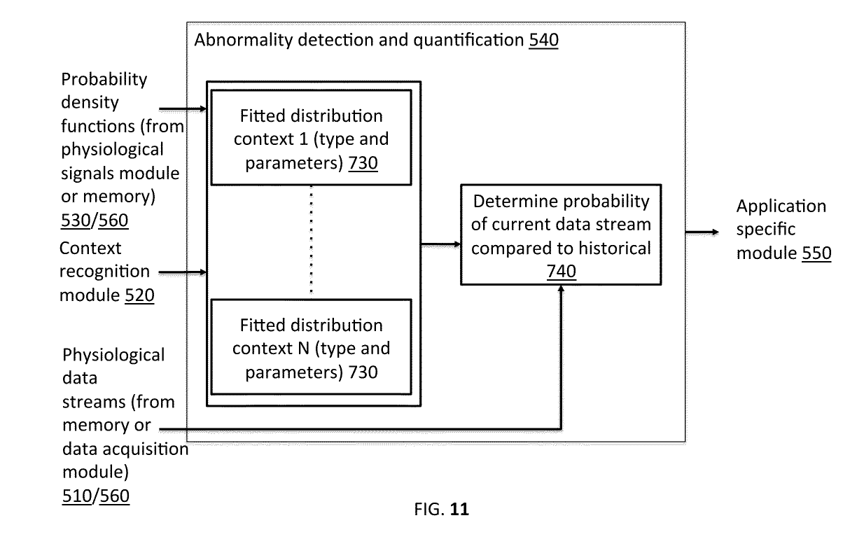

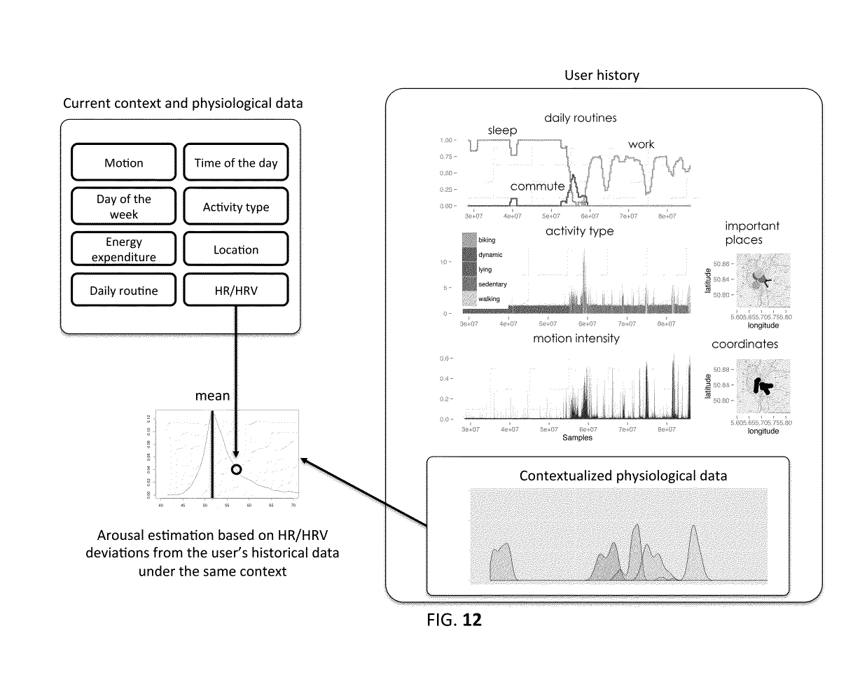

FIG. 2J is an exemplary flow chart illustrating a preferred embodiment for acquiring data of FIG. 1A or 1B wherein acquiring data includes receiving data from smartphone sensors, wearable sensors, ambient sensors, APIs and manual input and preprocessing data.

FIG. 3A is an exemplary flow chart illustrating a preferred embodiment for recognizing context of FIG. 1A or 1B wherein recognizing context includes recognizing activity type from the acquired data.

FIG. 3B is an exemplary flow chart illustrating a preferred embodiment for recognizing context of FIG. 1A or 1B wherein recognizing context includes recognizing activity type and detecting important places from the acquired data.

FIG. 3C is an exemplary flow chart illustrating a preferred embodiment for recognizing context of FIG. 1A or 1B wherein recognizing context includes recognizing activity type, detecting important places and detecting routines from the acquired data.

FIG. 3D is an exemplary flow chart illustrating a preferred embodiment for recognizing context of FIG. 1A or 1B wherein recognizing context includes recognizing activity type, detecting important places, detecting routines, estimating energy expenditure, detecting circadian rhythm and recognizing transportation mode from the acquired data.

FIG. 4 is an exemplary flow chart illustrating a preferred embodiment for modeling physiological signals of FIG. 1A or 1B wherein modeling physiological signals includes fitting distribution using recognized context of FIG. 3A-D and acquired physiological data of FIG. 2A-2J.

FIG. 5 is an exemplary flow chart illustrating a preferred embodiment for detecting and quantifying abnormalities of FIG. 1A or 1B.

FIG. 6A is an exemplary top-level block diagram illustrating an embodiment of a system for detecting deviations of the physiological signals normality of a person, wherein the system includes data acquisition, context recognition and physiological signals modeling.

FIG. 6B is an exemplary block diagram illustrating an alternative embodiment of the system of FIG. 6A, wherein the system further includes abnormality detection and quantification.

FIG. 6C is an exemplary block diagram illustrating an alternative embodiment of the system of FIG. 6A, wherein the system is used for specific applications.

FIG. 6D is an exemplary block diagram illustrating an alternative embodiment of the system of FIG. 6A, wherein the system is used for specific applications and further includes a memory module.

FIG. 7A is an exemplary block diagram illustrating a preferred embodiment of a Data Acquisition Module of FIG. 6A or 6B or 6C or 6D wherein the data acquisition module includes data streams from smartphone sensors.

FIG. 7B is an exemplary block diagram illustrating a preferred embodiment of a Data Acquisition Module of FIG. 6A or 6B or 6C or 6D wherein the data acquisition module includes data streams from smartphone sensors and signal preprocessing.

FIG. 7C is an exemplary block diagram illustrating a preferred embodiment of a Data Acquisition Module of FIG. 6A or 6B or 6C or 6D wherein the data acquisition module includes data streams from wearable sensors.

FIG. 7D is an exemplary block diagram illustrating a preferred embodiment of a Data Acquisition Module of FIG. 6A or 6B or 6C or 6D wherein the data acquisition module includes data streams from wearable sensors and signal preprocessing.

FIG. 7E is an exemplary block diagram illustrating a preferred embodiment of a Data Acquisition Module of FIG. 6A or 6B or 6C or 6D wherein the data acquisition module includes data streams from smartphone sensors and wearable sensors.

FIG. 7F is an exemplary block diagram illustrating a preferred embodiment of a Data Acquisition Module of FIG. 6A or 6B or 6C or 6D wherein the data acquisition module includes data streams from smartphone sensors and wearable sensors and signal preprocessing.

FIG. 7G is an exemplary block diagram illustrating a preferred embodiment of a Data Acquisition Module of FIG. 6A or 6B or 6C or 6D wherein the data acquisition module includes data streams from smartphone sensors, wearable sensors and manual input.

FIG. 7H is an exemplary block diagram illustrating a preferred embodiment of a Data Acquisition Module of FIG. 6A or 6B or 6C or 6D wherein the data acquisition module includes data streams from smartphone sensors, wearable sensors and manual input and signal preprocessing.

FIG. 7I is an exemplary block diagram illustrating a preferred embodiment of a Data Acquisition Module of FIG. 6A or 6B or 6C or 6D wherein the data acquisition module includes data streams from smartphone sensors, wearable sensors, APIs, ambient sensors and manual input.

FIG. 7J is an exemplary block diagram illustrating a preferred embodiment of a Data Acquisition Module of FIG. 6A or 6B or 6C or 6D wherein the data acquisition module includes data streams from smartphone sensors, wearable sensors, APIs, ambient sensors and manual input and signal preprocessing.

FIG. 8A is an exemplary block diagram illustrating a preferred embodiment for a Context Recognition Module of FIG. 6A or 6B or 6C or 6D wherein the context recognition module includes activity recognition.

FIG. 8B is an exemplary block diagram illustrating a preferred embodiment for a Context Recognition Module of FIG. 6A or 6B or 6C or 6D wherein the context recognition module includes activity recognition and important place detection.

FIG. 8C is an exemplary block diagram illustrating a preferred embodiment for a Context Recognition Module of FIG. 6A or 6B or 6C or 6D wherein the context recognition module includes activity recognition, important place detection and routine detection.

FIG. 8D is an exemplary block diagram illustrating a preferred embodiment for a Context Recognition Module of FIG. 6A or 6B or 6C or 6D wherein the context recognition module includes activity recognition, important place detection, routine detection, energy expenditure estimation, circadian rhythm detection and transportation mode recognition.

FIG. 9 shows schematically an example of the context recognition module where different contextual information are derived from data acquired by the data acquisition module.

FIG. 10 is an exemplary block diagram illustrating an embodiment of the physiological signals modeling module.

FIG. 11 is an exemplary block diagram illustrating an embodiment of the abnormality detection and quantification module.

FIG. 12 is an exemplary block diagram illustrating an embodiment of the physiological stress detection example, including the data streams.

It should be noted that the figures are not drawn to scale and that elements of similar structures or functions are generally represented by like reference numerals for illustrative purposes throughout the figures. It also should be noted that the figures are only intended to facilitate the description of the preferred embodiments. The figures do not illustrate every aspect of the described embodiments and do not limit the scope of the present disclosure.

### DETAILED DESCRIPTION OF THE PREFERRED EMBODIMENTS

Since currently-available health-related applications rely on supervised methods and require a significant amount of data from other persons, a biometric and context based method and system that detects and quantifies deviations from physiological signals normality, can prove desirable for a wide range of applications and/or medical conditions to allow for lifestyle management and/or monitoring of chronic medical conditions. This can be achieved, according to one embodiment disclosed herein, by the method 100 for biometric and context based messaging as illustrated in FIG. 1A or FIG. 1B.

Turning to FIG. 1A, advantageously, the method 100 can provide results relative to the norms of the subject continuously in free-living conditions, thus overcoming conventional limitations relying on population-derived norms and on limited, low level or no context. Additionally and/or alternatively, the method 100 advantageously can provide a quantification of the extent to which physiological signals deviates from the norms of the subject. A subject can be a human, a non-human animal, or a plant.

Advantageously, the method 100 can determine and quantify subject-specific deviations from physiological signals of the subject without a need for population-derived data. This is advantageous because each subject physiology differs and creating individual models of the physiology of a subject allows for very precise understanding of the specific conditions of the subject. Precise interpretation of the subject specific conditions is not possible when using population-derived norms. Additionally, the method 100 advantageously does not require stratifying subjects based on anthropometric characteristics (e.g. body weight, height, body fat, age, etc.) since norms are specific to a subject. Additionally, the method is able to determine the subject context. Determining the subject context is advantageous because physiological data can be interpreted continuously providing richer information compared to single spot checks. Further advantageously, the physiological signals models can vary over places, activities, time and environmental factors when determining the subject context. Additionally, the method is able to create a model of the physiological signals of the subject distribution in each different context. Advantageously, one or more model of the physiological signals of the subject can be determined. Each model of the physiological signals distributions of the subject in each different context defines the own norms of the subject. Advantageously, the method can provide quantifications of the degree to which the physiological signals are deviating from the own norms of the subject. This is advantageous because quantified information can be used to enable different responses, for example triggering different levels of alerts.

Method 100

The present disclosure concerns a method for detecting and quantifying deviations from physiological signal normality. This can be achieved, according to one embodiment disclosed herein, by the method of FIG. 1A. As illustrated in FIG. 1A, the method 100 comprises acquiring data, at 110, recognizing context, at 120, and modeling physiological signals, at 130. The data can be acquired, at 110, in any conventional manner. The data, for example, can comprise biometric and contextual data that is received from one or more sensors (not shown). The sensors can include sensors that measure uniform and/or different types of data. In one embodiment, a first sensor can measure biometric data, and a second sensor can measure contextual data. Exemplary biometric data can include heart rate value, heart rate variability values, blood pressure values, galvanic skin response level value, and/or respiration value data. Data regarding motion intensity and/or other accelerometer data, coordinates, and/or anthropometric characteristics can also be included. Acquired data is used for example to recognize context. Context, at 120, can be recognized from the raw data and include information related to the activity type, routine, important places, etc. of the subject. Modeling physiological signals, at 130, can compute single and multi-parameter distributions of physiological signals, acquired using acquiring data, for different contexts determined using recognizing context. Physiology in context can be further analyzed using detecting abnormality, at 140, which detects and quantifies physiological deviations from the norms of a subject, as determined by modeling physiological signals.

In another embodiment, illustrated on FIG. 1B, the method for detecting and quantifying deviations from physiological signal normality in FIG. lA further includes detecting and quantifying abnormalities, at 140.

Acquiring Data 110

Acquiring data, at 110, refers to acquiring data for method 100 in any conventional manner. The data, for example, can comprise biometric and contextual data. In the manner discussed above with reference to FIG. 1A, the exemplary biometric data can include heart rate value, heart rate variability values, blood pressure values, galvanic skin response level value, and/or respiration value data. Data regarding motion intensity and/or other accelerometer data, coordinates, and/or anthropometric characteristics can also be included. Acquiring data, at 110, can consist in receiving data streams from sensors and optionally preprocessing data streams. The term “sensor” has to be construed in its broadest and most general meaning as a means for measuring, including but not limited to sensors producing waveforms representing biological, physiological, neurological, psychological, physical, chemical, electrical and mechanical signals, such as pressure, sound, temperature and the like, probes, surveillance equipment, measuring equipment, and any other means for monitoring parameters representative of or characteristic for an application domain. When applied to the field of wellness, lifestyle, health or healthcare, the sensors are typically used to measure body signals and the surrounding environment. The sensors may sense physical, and/or physiological and/or environmental signals. The one or many sensors may be embedded in a smartphone (smartphone sensors), be embedded in the environment (ambient sensors), be carried by the user (ambulatory sensor), and/or be worn by the user (wearable sensors). Additionally, sensors of different kinds (smartphone, ambient and wearable sensors) might be accessed via the internet by means of Application Programming Interfaces (APIs) which allow to acquire additional data streams such as for example data coming from third party wearable sensors or third party ambient sensors such as weather stations. Smart phone sensors may include, but are not limited to: accelerometers, camera, Light Emitting Diode (LED), microphone. For the purpose of this disclosure, GPS, Bluetooth and other wireless communication means may also be considered as sensors. Ambient sensors may include, but are not limited to, environment temperature and humidity, PIR sensors or ultrasounds for movement detection or people counting, etc. Wearable sensors may include, but are not limited to, blood pressure monitors, blood glucose monitors, biochemical assays to measure specific hormones such as cortisol, melatonin, etc., activity trackers, heart rate trackers (chest strap or wrist based), ECG monitors, EEG monitors, etc. Information about the physiological signals of the subject can be received from a heart beat rate (HR) or electrocardiogram (ECG) sensor in case of heart rate, from a wrist, arm-band or ankle-band in case of galvanic skin response (GSR), or generally speaking a body patch able to acquire information about, for example, ECG, HR, GSR, skin temperature, skin humidity, respiration or other physiological signal and may be information about current or real-time value of one or more of the above mentioned physiological signals for the subject.

Turning to FIG. 2A, acquiring data 110 can include receiving data from data streams 210, and receiving data from data streams 210 can further include receiving data streams from smartphone sensors 211. Turning to FIG. 2B, alternatively, acquiring data 110, can consist in receiving data streams from smartphone sensor 211, and performing signal preprocessing 220. Performing signal preprocessing 220, can include filtering data 221, and correcting artifacts in data 222. The signal pre-processing component 220, can pre-process signals to generate pre-processed signals. The pre-processing 220, may include combining signals provided by the sensor into one or multiple meaningful composite signals. Turning to FIG. 2C, acquiring data 110 can consist in receiving data streams from wearable sensors 212. Turning to FIG. 2D, alternatively, acquiring data 110 can consist in receiving data streams from wearable sensors 212 and performing signal preprocessing 220. Performing signal preprocessing 220 can include filtering data 221 and correcting artifacts in data 222. Turning to FIG. 2E, acquiring data 110 can consist in receiving data streams from smartphone sensors 211 and receiving data streams form wearable sensors 212. Turning to FIG. 2F, alternatively, acquiring data 110 can consist in receiving data streams from smartphone sensors 211 and receiving data streams from wearable sensors 212 and performing signal preprocessing 220. Performing signal preprocessing 220 can include filtering data 221 and correcting artifacts in data 222. Turning to FIG. 2G, acquiring data 110 can consist in receiving data streams from smartphone sensors 211 receiving data streams from wearable sensors 212 and receiving data streams from manual input 213. Turning to FIG. 2H, alternatively, acquiring data 110 can consist in receiving data streams from smartphone sensors at 211 receiving data streams from wearable sensors 212 and receiving data streams from manual input 213 and performing signal preprocessing 220. Performing signal preprocessing 220 can include filtering data 221 and correcting artifacts in data 222. Turning to FIG. 2I, acquiring data 110 can consist in receiving data streams from smartphone sensors 211, receiving data streams from wearable sensors 212, receiving data streams from manual input 213, receiving data streams from ambient sensors 214 and receiving data streams from APIs 215. Turning to FIG. 2J, alternatively, acquiring data 110 can consist in receiving data streams from smartphone sensors 211, receiving data streams from wearable sensors 212, receiving data streams from manual input 213, receiving data streams from ambient sensors 214, and receiving data streams from APIs 215, and performing signal preprocessing 220. Performing signal preprocessing 220, can include filtering data 221 and correcting artifacts in data 222.

Recognizing Context 120

Turning to FIG. 3A, recognizing context 120 refers to recognizing context after acquiring data 110 in any conventional manner. Acquiring data 110 results in data streams 210 which can include but are not limited to GPS traces or other coordinates (e.g. acquired from WiFi or GSM networks), accelerometer data or other inertial unit data, physiological data and other data streams. Anthropometric characteristics can also be included via manual input or other manners. Turning to FIG. 3A, recognizing context 120 can consist in recognizing activity 310. Exemplary recognized activities can include one or more of: lying, sedentary behavior, household and dynamic/mixed activities, walking, biking, running and other sports activities. The activities might be related or mutually exclusive. The activities can be recognized by using pattern recognition techniques applied on the accelerometer data and/or coordinates and/or anthropometric characteristics and/or physiological data streams. Examples of mathematical models used by the activity recognition component can include Support Vector Machines, decision trees, neural networks, naïve Bayes or other classifiers. The coefficients and/or parameters of the models can be derived, for example, using information about activity performed, acceleration, coordinates, physiological data and anthropometric characteristics in different environments and while a number of subjects is performing a range of activities. This procedure is often referred to as supervised learning; however, unsupervised learning techniques could also be used.

Alternatively, FIG. 3B illustrates recognizing context by recognizing activity 310 and detecting important places 320. Detecting important places 320 can consist in determining, calculating, and/or detecting from information received about the coordinates of the subject, the stay regions of the subject. The stay regions are the locations in which the subject spends most of his/her more frequently.

Alternatively, FIG. 3C illustrates recognizing context by recognizing activity 310, detecting important places 320 and detecting routines 330. Detecting routines 330 can consist in using acquired data such as motion intensity or other accelerometer features, coordinates or other ways of detecting the location of the subject, and other data such as time information. Such input information can be used to determine, calculate and/or detect the daily routines of the subject, such as being at home, working, having lunch, commuting, etc. Daily routines are high-level interpretations of activities of the subject, able to capture contextual information not available at the lower level of the activity recognition system. For example, the recognized activities can be sedentary activities, walking and transitions between activities, which can take place in different daily routines, for example while working or while spending time relaxing at home. Advantageously, detecting daily routines 330 can allow for inference of higher-level context, which is advantageous to detect and quantify deviations from the physiological data normality of a subject. Daily routines can be detected in an unsupervised manner since each subject is different and performs different high level activities. Daily routines can be detected using pattern recognition techniques, which can be unsupervised and applied to the accelerometer data or other inertial data and optionally on coordinates and/or other information such as time. Examples of mathematical models used by the daily routine detection module are for example Latent Dirichlet Allocation or other parametric and non-parametric topic models. The coefficients and or parameters of the models can be derived, for example, using information about activity performed, acceleration, coordinates, anthropometric characteristics in different environments and while the subject is performing a range of daily routines over a number of days without requiring reference training data therefore in an unsupervised manner.

Alternatively, FIG. 3D illustrates recognizing context by recognizing activity 310, detecting important places 320, detecting routines 330, estimating energy expenditure 340, detecting circadian rhythm 350 and recognizing transportation modes 360. Estimating energy expenditure 340 can be performed in two steps. First, an activity can be recognized by recognizing activity 310. Secondly, an activity-specific energy expenditure model can be applied to derive energy expenditure. An activity-specific energy expenditure model can be a multiple linear or non-linear regression model developed using motion information, physiological signals information, anthropometric characteristics and reference oxygen consumption from a number of subjects performing the specific activity. A different model can be used for each detected activity in order to capture the peculiar relation between an activity and energy expenditure during an activity. Detecting circadian rhythm 350 can be performed receiving input about the motion intensity or other accelerometer features and/or physiological data as well as activities for a subject. The received input is used to determine, calculate and/or detect the individual circadian rhythm of a subject. An example of the circadian rhythm of a subject is the sleep-wake individual cycle. Recognizing transportation modes 360 consists in determining, calculating, and/or detecting from information received about the coordinates of a subject, the transportation mode of the subject. Recognizing transportation modes 360 consists in determining, calculating and/or detecting at least two transportation modes. Exemplary transportation modes include “automotive” and “active transportation”. Automotive can refer to transportation modes such as by car, by train, by bus and/or by plane; while, active transportation can refer to transportation by bike, walking, running or other active transportation mode. Examples of mathematical models used by the transportation mode recognition component include Support Vector Machines, decision trees, neural networks, naïve Bayes or other classifiers. The coefficients and/or parameters of the models can be derived, for example, using information about transportation mode, activity performed, acceleration, coordinates, anthropometric characteristics in different environments and while a number of subjects is involved in a range of transportation modes.

Modeling Physiological Signals 130

Turning to FIG. 4, modeling physiological data 130 refers to creating a model of the physiology of a subject in a particular context 410. Such modeling of physiological signals can, for example, consist in fitting a distribution 420. Fitting a distribution 420 can consist in fitting a multi-parameter distribution of physiological signals data if more than one physiological signal is acquired. Alternatively, fitting a distribution 420 it can be a single-parameter distribution if a single physiological signal is acquired.

According to an embodiment, the single or multi-parameter distribution or probability density function created at 420 can be obtained using parametric or non-parametric methods. Exemplary probability density function include histograms or more advanced methods such as kernel density estimations used to make inferences on the population distribution based on a sample of data. According to an embodiment, the single or multi-parameter distribution of physiological signals in a context can represent the probability of each physiological signal or combination of physiological signals to assume certain values in certain contexts 410. Advantageously, stratification based on anthropometric is not required, since norms are specific of a subject as defined by its own physiological values over an historical period of time. Advantageously, the single or multi-parameter distribution of physiological signals in a context allows for detection and quantification of abnormalities in physiological data. The physiological modeling 130 does not need to rely on large stratified databases collected on a population with similar conditions, which might be impossible to acquire. Additionally, modeling physiological signals 130 allows detecting and quantifying abnormalities for subjects having values within so-called population norms, which are often too broad and cause early conditions to be overlooked. An exemplary condition is hypertension, which can be overlooked by too broad blood pressure population norms. According to an embodiment, the single or multi-parameter distribution or probability density function of physiological signals in a context can be named the historical distribution or historical probability density function of physiological signals for an individual. A different historical distribution for each context can be determined. Advantageously, the historical distribution can be of different durations, to allow for acquiring enough data to model the signal distribution but also account for changes in time due to other factors for example aging.

Detecting and Quantifying Abnormalities 140

Turning to FIG. 5, detecting and quantifying abnormalities 140 can consist in determining the probability of the current data stream compared to a fitted distribution 450. Detecting and quantifying abnormalities 140 can be performed, for example, by computing the probability of the physiological signals of a subject in a certain context. The probability of the physiological signal of the subject in a certain context is given by the historical distribution of physiological signals in the same context. According to an embodiment, detecting and quantifying abnormalities 140 requires as input the distribution type and parameters in a specific context as determined when modeling physiological data 420. Additional input can be the physiological signals 130 to be classified as belonging to the user norms or abnormal, as received when acquiring data 110 and the current recognized context 120.

According to an embodiment, the physiological signals deviations from the norms of the subject can be quantified by the degree to which the physiological signal data differs from the historical distribution. The historical distribution of values is modeled as a probability density function with known parameters as fitted modeling physiological data 130. Deviations from such probability density functions in terms of differences between the real-time or non-real-time values to be classified and such probability density function can be interpreted as being within norms or abnormalities that can after be quantified 450. The current recognized context can be used to determine which distribution type and set of parameters are to be used by when detecting and quantifying abnormalities 140. Furthermore, once the fitted distribution has been selected, the current physiological data streams, as received when acquiring data, can be compared against the probability density function of historical data to determine and quantify abnormalities.

System 500

The present disclosure also concerns a system for detecting and quantifying deviations from physiological signal normality. This can be achieved, according to one embodiment disclosed herein, by the system of FIG. 6A. As illustrated in FIG. 6A, the system 500 comprises a data acquisition module, at 510, a context recognition module, at 520, and a physiological signals modeling module, at 530. The data acquisition module 510 can acquire data in any conventional manner. The data, for example, can comprise biometric and contextual data that is received from one or more sensors (not shown). The sensors can include sensors that measure uniform and/or different types of data. In one embodiment, a first sensor can measure biometric data, and a second sensor can measure contextual data. Exemplary biometric data can include heart rate value, heart rate variability values, blood pressure values, galvanic skin response level value, and/or respiration value data. Data regarding motion intensity and/or other accelerometer data, coordinates, and/or anthropometric characteristics can also be included. The context recognition module 520 can be used to analyze the acquired data and extract contextual information related to the activity type, routine, important places, etc. of the subject. The physiological signals modeling module 530 can compute single and multi-parameter distributions of physiological signals, acquired using the data acquisition module, for different contexts determined using the context recognition module.

In another embodiment, illustrated on FIG. 6B, the system for detecting and quantifying deviations from physiological signal normality of FIG. 6A can further include an abnormality detection and quantification module 540.

In another embodiment, illustrated on FIG. 6C, the system for detecting and quantifying deviations from physiological signal normality of FIG. 6B can further include an application specific module 550.

In another embodiment, illustrated on FIG. 6D, the system for detecting and quantifying deviations from the physiological signal normality of a subject of FIG. 6C can further include a memory module 560.

Data Acquisition Module 510

Turning to FIG. 7A, the data acquisition module 510 can acquire data for the system 500. The data acquisition module 510 can acquire the data in any conventional manner. The data, for example, can comprise biometric and contextual data. The exemplary biometric data can include heart rate value, heart rate variability values, blood pressure values, galvanic skin response level value, and/or respiration value data. Data regarding motion intensity and/or other accelerometer data, coordinates, and/or anthropometric characteristics can also be included. The data acquisition module 510 can consist of a data streams component 520, receiving data streams from sensors, and a signal preprocessing component 530. The term “sensor” has to be construed in its broadest and most general meaning as a means for measuring, including but not limited to sensors producing waveforms representing biological, physiological, neurological, psychological, physical, chemical, electrical and mechanical signals, such as pressure, sound, temperature and the like, probes, surveillance equipment, measuring equipment, and any other means for monitoring parameters representative of or characteristic for an application domain. When applied to the field of wellness, lifestyle, health or healthcare, the sensors are typically used to measure body signals and the surrounding environment. The sensors may sense physical, and/or physiological and/or environmental signals. The one or many sensors may be embedded in a smartphone (smartphone sensors), be embedded in the environment (ambient sensors), be carried by the user (ambulatory sensor), and/or be worn by the user (wearable sensors). Additionally, sensors of different kinds (smartphone, ambient and wearable sensors) might be accessed via the internet by means of Application Programming Interfaces (APIs) which allow to acquire additional data streams such as for example data coming from third party wearable sensors or third party ambient sensors such as weather stations. Smart phone sensors may include, but are not limited to: accelerometers, camera, Light Emitting Diode (LED), microphone. For the purpose of this disclosure, GPS, Bluetooth and other wireless communication means may also be considered as sensors. Ambient sensors may include, but are not limited to, environment temperature and humidity, PIR sensors or ultrasounds for movement detection or people counting, etc. Wearable sensors may include, but are not limited to, blood pressure monitors, blood glucose monitors, biochemical assays to measure specific hormones such as cortisol, melatonin, etc., activity trackers, heart rate trackers (chest strap or wrist based), ECG monitors, EEG monitors, etc. Information about the physiological signals of the subject can be received from a heart beat rate (HR) or electrocardiogram (ECG) sensor in case of heart rate, from a wrist, arm-band or ankle-band in case of galvanic skin response (GSR), or generally speaking a body patch able to acquire information about, for example, ECG, HR, GSR, skin temperature, skin humidity, respiration or other physiological signal and may be information about current or real-time value of one or more of the above mentioned physiological signals for the subject.

Turning to FIG. 7A, the data acquisition module 510 can include a data streams component 520 acquiring data from smartphone sensors 521.

Turning to FIG. 7B, alternatively, the data acquisition module 510 can include a data streams component 520 acquiring data from smartphone sensors 521 and a signal preprocessing component 530 performing signal preprocessing. Performing signal preprocessing can include a filter component 531 and an artifacts correction component 532. The signal pre-processing component 530 can pre-process signals to generate pre-processed signals. The pre-processing may include combining signals provided by the sensor into one or multiple meaningful composite signals.

Turning to FIG. 7C, the data acquisition module 510 can include a data streams component 520 acquiring data from wearable sensors 522.

Turning to FIG. 7D, alternatively, the data acquisition module 510 can include a data streams component 520 acquiring data from wearable sensors 522 and a signal preprocessing component 530 performing signal preprocessing on the acquired data.

Turning to FIG. 7E, the data acquisition module 510 can include a data streams component 520 acquiring data from smartphone sensors 521 and wearable sensors 522.

Turning to FIG. 7F, alternatively, the data acquisition module 510 can include a data streams component 520 acquiring data from smartphone sensors 521 and wearable sensors 522 and a signal preprocessing component 530 performing signal preprocessing on the acquired data.

Turning to FIG. 7G, the data acquisition module 510 can include a data streams component 520 acquiring data from smartphone sensors 521, wearable sensors 522 and manual input 523.

Turning to FIG. 7H, alternatively, the data acquisition module 510 can include a data streams component 520 acquiring data from smartphone sensors 521, wearable sensors 522 and manual input 523 and a signal preprocessing component 530 performing signal preprocessing on the acquired data.

Turning to FIG. 7I, the data acquisition module 510 can include a data streams component 520 acquiring data from smartphone sensors 521, wearable sensors 522, manual input 523, ambient sensors 524 and APIs 525.

Turning to FIG. 7H, alternatively, the data acquisition module 510 can include a data streams component 520 acquiring data from smartphone sensors 521, wearable sensors 522, manual input 523, ambient sensors 524 and APIs 525 and a signal preprocessing component 530 performing signal preprocessing on the acquired data. The system may be attached to the body of the subject or separated from it, for example, implemented in a separate mobile unit or integrated in a mobile phone.

Context Recognition Module 520

Turning to FIG. 8A, recognizing context, at 120, can be performed using a context recognition module 520, wherein the context recognition module can receive pre-processed data or raw data from the data acquisition module 510. The data can include but is not limited to GPS traces or other coordinates (e.g. acquired from WiFi or GSM networks), accelerometer data or other inertial unit data, physiological data and other data streams. Anthropometric characteristics can also be included via of a user interface with manual input or other manners. The context recognition module 520 can include one or more components. Turning to FIG. 8A, the context recognition module can include an activity recognition component 610. Exemplary recognized activities can include one or more of: lying, sedentary behavior, household and dynamic/mixed activities, walking, biking, running and other sports activities. The activities might be related or mutually exclusive. The activities can be recognized by using pattern recognition techniques applied on the accelerometer data and/or coordinates and/or anthropometric characteristics and/or physiological data streams. Examples of mathematical models used by the activity recognition component can include Support Vector Machines, decision trees, neural networks, naïve Bayes or other classifiers. The coefficients and/or parameters of the models can be derived, for example, using information about activity performed, acceleration, coordinates, physiological data and anthropometric characteristics in different environments and while a number of living beings is performing a range of activities. This procedure is often referred to as supervised learning; however, unsupervised learning techniques could also be used.

Alternatively, FIG. 8B illustrates another preferred embodiment of the context recognition module 520 including an activity recognition component 610 and an important place detection component 620. The important place detection component 620 determines, calculates, and/or detects from information received about the coordinates of the subject, the stay regions of the subject. The stay regions are the locations in which the subject spends most of his/her more frequently.

Alternatively, FIG. 8C illustrates another preferred embodiment of the context recognition module 520, where the context recognition module 520 includes an activity recognition component 610, an important place detection component 620 and a routine detection component 630. The routine detection component 630 uses acquired data such as motion intensity or other accelerometer features, coordinates or other ways of detecting the location of the subject, and other data such as time information. Such input information is used to determine, calculate and/or detect the daily routines of the subject, such as being at home, working, having lunch, commuting, etc. Daily routines are high-level interpretations of activities of the subject, able to capture contextual information not available at the lower level of the activity recognition system. For example, the recognized activities can be sedentary activities, walking and transitions between activities, which can take place in different daily routines, for example while working or while spending time relaxing at home. Advantageously, detecting daily routines can allow for inference of higher-level context, which is advantageous to detect and quantify deviations from the physiological data normality of a subject. Daily routines can be detected in an unsupervised manner since each subject is different and performs different high level activities. Daily routines can be detected using pattern recognition techniques, which can be unsupervised and applied to the accelerometer data or other inertial data and optionally on coordinates and/or other information such as time. Examples of mathematical models used by the daily routine detection module are for example Latent Dirichlet Allocation or other parametric and non-parametric topic models. The coefficients and or parameters of the models can be derived, for example, using information about activity performed, acceleration, coordinates, anthropometric characteristics in different environments and while the subject is performing a range of daily routines over a number of days without requiring reference training data therefore in an unsupervised manner.

Alternatively, FIG. 8D illustrates another preferred embodiment of the context recognition component 520, including an activity recognition component 610, an important place detection component 620, a routine detection component 630, an energy expenditure component 640, a circadian rhythm detection component 650 and a transportation mode recognition component 660. The energy expenditure estimation component 640 estimates energy expenditure in two steps. First, an activity can be recognized by using the activity recognition component. Secondly, an activity-specific energy expenditure model can be applied to derive energy expenditure. An activity-specific energy expenditure model can be a multiple linear or non-linear regression model developed using motion information, physiological signals information, anthropometric characteristics and reference oxygen consumption from a number of subjects performing the specific activity. A different model can be used for each detected activity in order to capture the peculiar relation between an activity and energy expenditure during an activity. The circadian rhythm detection component 650 receives input about the motion intensity or other accelerometer features and/or physiological data as well as activities as recognized by the activity recognition component for a subject. The received input is used to determine, calculate and/or detect the individual circadian rhythm of a subject. An example of the circadian rhythm of a subject is the sleep-wake individual cycle. The transportation mode recognition component 660 can determine, calculate, and/or detect from information received about the coordinates of a subject, the transportation mode of the subject. Recognizing transportation mode consists of determining, calculating and/or detecting at least two transportation modes. Exemplary transportation modes include “automotive” and “active transportation”. Automotive can refer to transportation modes such as by car, by train, by bus and/or by plane; while, active transportation can refer to transportation by bike, walking, running or other active transportation mode. Examples of mathematical models used by the transportation mode recognition component include Support Vector Machines, decision trees, neural networks, naïve Bayes or other classifiers. The coefficients and/or parameters of the models can be derived, for example, using information about transportation mode, activity performed, acceleration, coordinates, anthropometric characteristics in different environments and while a number of subjects is involved in a range of transportation modes.

According to an embodiment, the context recognition module 520 can compute, manage and store the physiological signal values obtained by the data acquisition module 510 and associated with each of the detected activities, daily routines, important places, transportation modes and other detected contexts for retrieval by the physiological signals modeling module 530. Such couples or tuples, including physiological signals and contexts as well as other contextual information, can be optionally stored in memory means such as a local database or in the cloud. An example of the Context Recognition module output including raw data streams is shown in FIG. 9. Motion intensity from accelerometer data and coordinates are examples of data streams 520 received by the data acquisition module 510. Activity type, daily routines and important places are examples of context derived by the components of the context recognition module 520.

Physiological Signals Modeling Module 530

Turning to FIG. 10, the method 100 can create a model of the physiology of a subject in a context. Such modeling of physiological signals, at 130, can be performed by the physiological signals modeling module 530. Such modeling of physiological signals can be, for example, the multi-parameter distribution 730 of physiological signals data 720 if more than one physiological signal is acquired. Alternatively, it can be a single-parameter distribution 730 if a single physiological signal is acquired 720. According to an embodiment, the single or multi-parameter distribution or probability density function 730 can be obtained using parametric or non-parametric methods. Exemplary probability density function include histograms or more advanced methods such as kernel density estimations used to make inferences on the population distribution based on a sample of data. According to an embodiment, the single or multi-parameter distribution of physiological signals in a context can represent the probability of each physiological signal or combination of physiological signals to assume certain values in certain contexts 710. Advantageously, stratification based on anthropometric is not required, since norms are specific of a subject as defined by its own physiological values over an historical period of time. Advantageously, the single or multi-parameter distribution of physiological signals in a context allows for detection and quantification of abnormalities in physiological data. The physiological modeling does not need to rely on large stratified databases collected on a population with similar conditions, which might be impossible to acquire. Additionally, modeling physiological signals allows detecting and quantifying abnormalities for subjects having values within so-called population norms, which are often too broad and cause early conditions to be overlooked. An exemplary condition is hypertension, which can be overlooked by too broad blood pressure population norms. According to an embodiment, the single or multi-parameter distribution or probability density function of physiological signals in a context can be named the historical distribution or historical probability density function of physiological signals for an individual and a different historical distribution for each context will be determined and stored. Advantageously, the historical distribution can be of different durations, to allow for acquiring enough data to model the signal distribution but also account for changes in time due to other factors for example aging.

According to an embodiment, the physiological signals modeling module 530 can compute, manage and/or store the probability density function type and parameters of the historical data assigned to each context obtained by the context recognition module 520. Such couples or tuples including physiological signals probability density functions types and parameters plus contexts as well as possibly other information can be optionally stored in memory systems such as a local database or in the cloud.

An example of physiological signals modeling for the case of stress detection can be the following. Assuming the physiological signal under consideration being heart rate and receiving such heart rate from the data acquisition module 510. The context recognition module 520 can provide a set of heart rate values matched to contexts, for example a set of heart rates while the subject is working and a set of heart rates while the subject being is sleeping. A moving time window over which the heart rate data are acquired can be for example a month for the case of stress detection. Once the physiological signals modeling module 530 receives as input the contextualized heart rate, an appropriate distribution is modeled by means of statistical measures. An example can be a normal distribution represented by the distribution parameters, such as the mean and standard deviation of the heart rate values in different contexts. The output of the physiological signals modeling module for the stress detection application can be the description of the type of distribution used to fit the data and the parameters defining such distribution.

In a related example, the device can be used to track stress during pregnancy. In this example, the moving time window over which data are acquired can typically be shorter than one month, such as for example, one week. The time window is different because physiological changes such as heart rate increase, naturally occur during pregnancy. A shorter time window makes it possible to avoid the influence of physiological changes naturally occurring during pregnancy in the stress detection application. In situations where physiological data is more stable, the time window constraint can be increased since more data can allow the context recognition module 520 to model more contexts.

Furthermore, once the fitted distribution has been selected, the current physiological data streams, as received from the data acquisition module 510 or the memory 560 can be compared against the probability density function of historical data to determine and quantify abnormalities.

An example of abnormality detection and quantification 540 for the case of the stress detection application can be, for example, based on the distance between the new values to be classified as stressful or non-stressful periods of time, and the historical probability density function (normal distribution of heart rates, as introduced in the previous section), in terms of standard deviation. Since by definition of normal distribution 95% of the distribution lies between two standard deviations, all new values could be classified as non-stressful when below two standard deviations. Abnormalities representing stressful events could be context-specific heart rate values more than two standard deviations greater than the mean of the probability density function of historical data. Alternatively, heart rate values differences from the mean of the probability density function of historical data could be divided by the standard deviation of the same probability density function to quantify deviations as values mainly belonging to the range 0 to 3, since 99% of the data lies into three standard deviations. Abnormality detection and quantification 540 allows for detection and quantification of abnormalities since a greater value (normalized by the standard deviation in case of a normal distribution as it is specified in this example) indicates greater abnormalities and higher stress level for the application of stress detection.

Abnormality Detection and Quantification Module 540

Turning to FIG. 11, detecting and quantifying abnormalities, at 140, can be performed by an abnormality detection and quantification module 540, wherein the abnormality detection and quantification module 540 can consist in determining, at 740, the probability of the current data stream 530 compared to a fitted distribution in a specific context 730. Detecting and quantifying deviations from physiological signals normality can be performed, for example, by computing the probability of the physiological signals of a subject in a certain context. The probability of the subject being physiological signal in a certain context is given by the historical distribution of physiological signals in the same context. According to an embodiment, detecting and quantifying abnormalities requires as input the distribution type and parameters in a specific context as determined when modeling physiological data 730. Additional input can be the physiological signals 530 to be classified as belonging to the user norms or abnormal, as received when acquiring data and the current recognized context 520.

According to an embodiment, the physiological signals deviations from the norms of the subject can be quantified by the degree to which the physiological signal data differs from the historical distribution. Since the historical distribution of values is modeled as a probability density function with known parameters as fitted modeling physiological data 530, deviations from such probability density functions in terms of differences between the real-time or non-real-time values to be classified and such probability density function can be interpreted as being within norms or abnormalities that can after be quantified 740. The current recognized context can be used to determine which distribution type and set of parameters are to be used by when detecting and quantifying abnormalities. Furthermore, once the fitted distribution has been selected, the current physiological data streams, as received when acquiring data, can be compared against the probability density function of historical data to determine and quantify abnormalities.

Application Specific Module 550

The application-specific module 550 in FIG. 6C can provide information related to a specific application such as stress detection or disease monitoring. For example in case of the stress detection application, the Application-Specific module 550 can provide feedback to the user, such as information about the stress level during the day. For the example of disease progression monitoring, the application-specific module 550 can provide either feedback to the subject or feedback to a doctor or other subject interested in monitoring the system user disease progression, for different reasons.

The method 100 and system 500 for detecting and quantifying deviations from a subject physiological signal normality described above is not limited to the specific scope of application and use described in the disclosed embodiments, and can extend to other applications and fields. For example, it shall be noted that other applications can be envisaged which may use the detection and quantification of abnormalities related to physiological or non-physiological signals to calculate another application-specific parameter associated to the subject. The method could also extend beyond living beings. For example the method can be applied to detect and quantify deviations from signal normality in the industrial space, on a manufacturing process line or in a manufacturing plan, to identify deviations to the norm that may be an indication of process flaws or issues. The method can also be applied to the field of agriculture, to identify deviations to the very controlled conditions of green houses for instance.

The functions of the modules described in FIGS. 6A to 11, according to the embodiments of the present disclosure, may be implemented using convention hardware and/or software system Exemplary hardware systems can include a conventional microcontroller, digital signal processor and/or any other multi-purpose integrated circuit comprising instruction processing capability, interface circuitry and memory. The system can be locally present on the device and/or located remotely from the device. In one embodiment, the hardware system can be in the cloud.

The disclosed embodiments are susceptible to various modifications and alternative forms, and specific examples thereof have been shown by way of example in the drawings and are herein described in detail. It should be understood, however, that the disclosed embodiments are not to be limited to the particular forms or methods disclosed, but to the contrary, the disclosed embodiments are to cover all modifications, equivalents, and alternatives.

What is claimed is:

1. A method for detecting a deviation from physiological normality, comprising:
acquiring physiological data and at least one of physical data and environmental data from a sensor associated with a subject device;
extracting contextual data from the acquired physical data and environmental data;
analyzing the physiological data with relevant contextual data to form an individualized physiological model;
comparing current physiological data and relevant current contextual data with the physiological model;
identifying the deviation based upon said comparing; and
reporting the deviation via the subject device.

2. The method of claim 1, wherein said analyzing the physiological data with the relevant contextual data includes generating the physiological model as a probability density function based upon historical physiological data and relevant historical contextual data, the probability density function establishing a normal physiological baseline for each of a plurality of selected contexts.

3. The method of claim 2,
wherein said acquiring physiological data includes acquiring a plurality of different physiological data types; and
wherein said generating the physiological model as the probability density function comprises generating a composite probability density function for a selected combination of the physiological data types.

4. The method of claim 3,
wherein said extracting contextual data includes extracting a plurality of different contextual data types; and
wherein said generating the composite probability density function comprises generating the composite probability density function for each of the contextual data types.

5. The method of claim 2,
wherein said acquiring physiological data includes acquiring a plurality of different physiological data types; and
wherein said generating the physiological model as the probability density function comprises generating a probability density function for each of the physiological data types.

6. The method of claim 2,
wherein said extracting contextual data includes extracting a plurality of different contextual data types; and
wherein said generating the physiological model as the probability density function comprises generating a probability density function for each of the contextual data types.

7. The method of claim 2,
wherein said generating the physiological model includes establishing an average value of the physiological data for each of the selected contexts; and
wherein said comparing comprises determining whether the current physiological data is within a predetermined range of the average value for the selected context corresponding to the relevant current contextual data.

8. The method of claim 7,
wherein said generating the physiological model includes establishing a standard deviation value of the physiological data for each of the selected contexts; and
wherein said determining whether the current physiological data is within the predetermined range comprises determining whether the current physiological data deviates from the average value by more than the standard deviation value for the selected context corresponding to the relevant current contextual data.

9. The method of claim 1, wherein said extracting the contextual data includes determining at least one of a set of mutually exclusive activities, a set of routines, a set of geographic locations, a set of energy expenditure levels, a set of circadian rhythms and a set of transportation modes each being relevant to the acquired physiological data.

10. The method of claim 1, further comprising analyzing the current physiological data with the relevant current contextual data to update the physiological model.

11. A computer program product for detecting a deviation from physiological normality, the computer program product being encoded on non-transitory machine-readable storage media and comprising:
instruction for acquiring physiological data and at least one of physical data and environmental data from a sensor associated with a subject device;
instruction for extracting contextual data from the acquired physical data and environmental data;
instruction for analyzing the physiological data with relevant contextual data to form an individualized physiological model;
instruction for comparing current physiological data and relevant current contextual data with the physiological model;
instruction for identifying the deviation based upon a result of the comparison; and
instruction for reporting the deviation via the subject device.

12. The computer program product of claim 11, wherein said instruction for analyzing the physiological data with the relevant contextual data includes instruction for generating the physiological model as a probability density function based upon historical physiological data and relevant historical contextual data, the probability density function establishing a normal physiological baseline for each of a plurality of selected contexts.

13. The computer program product of claim 12,
wherein said instruction for acquiring physiological data includes instruction for acquiring a plurality of different physiological data types; and
wherein said instruction for generating the physiological model as the probability density function comprises instruction for generating a composite probability density function for a selected combination of the physiological data types.

14. The computer program product of claim 13,
wherein said instruction for extracting contextual data includes instruction for extracting a plurality of different contextual data types; and
wherein said instruction for generating the composite probability density function comprises instruction for generating the composite probability density function for each of the contextual data types.

15. The computer program product of claim 12,
wherein said instruction for acquiring physiological data includes instruction for acquiring a plurality of different physiological data types; and
wherein said instruction for generating the physiological model as the probability density function comprises instruction for generating a probability density function for each of the physiological data types.

16. The computer program product of claim 12,
wherein said instruction for generating the physiological model includes instruction for establishing an average value of the physiological data for each of the selected contexts; and
wherein said instruction for comparing comprises instruction for determining whether the current physiological data is within a predetermined range of the average value for the selected context corresponding to the relevant current contextual data.

17. A system for detecting a deviation from physiological normality, comprising:
a data acquisition module for acquiring physiological data and at least one of physical data and environmental data from a sensor;
a context recognition module for extracting contextual data from the acquired physical data and environmental data;
a physiological modeling module for analyzing the physiological data with relevant contextual data to form an individualized physiological model; and
an abnormality detection module for comparing current physiological data and relevant current contextual data with the physiological model and identifying the deviation based upon a result of the comparison.

18. The system of claim 17, wherein the sensor is selected from the group consisting of an accelerometer, a motion sensor, a Global Positioning System device, a camera, a light emitting diode, and an optical sensor.

19. The system of claim 17, wherein the sensor includes at least one wearable sensor for providing at least one of the physiological data, the physical data and the environmental data on a continuous basis.

20. The system of claim 17, further comprising an application-specific module for reporting a selected characteristic of the identified deviation over a predetermined time period.
